(19) United States
(12) Patent Application Publication
(10) Pub. No.: US 2023/0287343 A1
(43) Pub. Date: Sep. 14, 2023

(54) METHOD FOR GENERATING A THREE-DIMENSIONAL NEUROMUSCULAR ORGANOID IN VITRO

(71) Applicant: MAX-DELBRÜCK-CENTRUM FÜR MOLEKULARE MEDIZIN IN DER HELMHOLTZ-GEMEINSCHAFT, Berlin (DE)

(72) Inventor: Asimina GKOUTI, Berlin (DE)

(73) Assignee: MAX-DELBRÜCK-CENTRUM FÜR MOLEKULARE MEDIZIN IN DER HELMHOLTZ-GEMEINSCHAFT, Berlin (DE)

(21) Appl. No.: 18/015,489

(22) PCT Filed: Jul. 12, 2021
(86) PCT No.: PCT/EP2021/069325

§ 371 Date: Jan. 10, 2023

Related U.S. Application Data
(60) Provisional application No. 63/050,736, filed on Jul. 10, 2020.

(51) Int. Cl. C12N 5/0797; A61P 25/14

(52) U.S. Cl. CPC C12N 5/0623; A61P 25/14; C12N 2500/99; C12N 2501/115; C12N 2501/105; C12N 2501/12; C12N 2501/415

(57) ABSTRACT

A method for generating a three-dimensional neuromuscular organoid in vitro includes providing a first cell culture that includes disease-specific neuromesodermal progenitor cells, 30% to 90% of which co-express BRACHYURY/SOX2 and 10% to 70% of which co-express TBX6. The disease-specific neuromesodermal progenitor cells are cultivated in a first differentiation medium chosen from the group consisting of i) a non-supplemented serum-free cell culture medium and ii) a serum-free cell culture medium supplemented with at least one of a ROCK inhibitor, an activator of a growth factor signaling pathway, and an activator of an insulin signaling pathway. The first differentiation medium is replaced by a second differentiation medium within 1 to 3 days after cultivation start. The second differentiation medium is replaced by a non-supplemented serum-free cell culture medium within another 1 to 3 days. A three-dimensional neuromuscular organoid is obtained from the non-supplemented serum-free cell culture medium.

### CROSS-REFERENCE TO A RELATED APPLICATION

This application is a National Phase Patent Application of International Patent Application Number PCT/EP2021/069325, filed on Jul. 12, 2021, which claims priority of U.S. Patent Application No. 63/050,736, filed on Jul. 10, 2020.

### BACKGROUND

Aspects of the disclosure relate to a method for generating a three-dimensional neuromuscular organoid in vitro, to a neuromuscular organoid obtainable by such a method, to methods of studying the development and/or mechanism of a disease in vitro by analyzing such a neuromuscular organoid, and to a kit comprising a serum-free cell culture medium appropriate for generating such a three-dimensional neuromuscular organoid.

Three-dimensional (3D), self-organizing, in vitro tissue models called organoids have been developed for a range of human tissues including the retina, brain, spinal cord, intestine, kidney, liver and pancreas (Broutier et al., 2016; Eiraku and Sasai, 2012; Huch et al., 2015; Lancaster et al., 2013; Morizane et al., 2015; Pasca et al., 2015; Spence et al., 2011). Cellular interactions in a three dimensional space are important for achieving better self-organization of tissue architecture and, therefore, 3D organoids are generally considered as a promising approach for tissue and disease modelling (Brassard and Lutolf, 2019) and were used successfully to model human diseases where a single, specific, tissue is affected (Rowe and Daley, 2019).

Yet, the study of diseases affecting a process in which more than a single tissue participates, such as neuromuscular disorders, remains a challenge. Neuromuscular diseases are caused by functional defects of the nervous system, skeletal muscle or arise by defects of the neuromuscular junction (NMJ) (Sanes and Lichtman, 1999). The NMJ is a highly organized chemical synapse formed between motor neurons (MNs) and skeletal muscles and it contains an additional important cell type, the terminal Schwann cells. In some of these disorders, only the muscle or the neural components are initially affected, but in many cases it is difficult to identify the primary cause and cell type affected. Additionally, some diseases exhibit regional specificity. Prominent examples are a group of more than thirty different acquired and genetic diseases characterized by neuromuscular transmission defects. Many of these diseases are incompletely understood and without treatment. In addition, muscular dystrophies as well as motor neuron diseases can have secondary effects at the neuromuscular junction and they have mostly escaped experimental approaches (Engel, 2018; Nicolau et al., 2019).

Significant progress has been made in the generation of either spinal cord neurons or skeletal muscles in 2D culture systems (Sances et al., 2016; Maury et al., 2015; Chal et al., 2016; Chal et al., 2015;) and the development of 2D and 3D co-culture systems (Afshar Bakooshli et al., 2019; Machado et al., 2019; Maffioletti et al., 2018; Osaki et al., 2018; Santhanam et al., 2018;

Steinbeck et al., 2016). However, no study has thus far succeeded in simultaneously generating all the components of the NMJ including terminal Schwann cells, that are essential for the maturation and support of NMJs (Darabid et al., 2014).

The generation of neuromuscular organoids from healthy neuromesodermal progenitor cells and their potential to be used as a model for neuromuscular diseases was recently described (Martins et al., 2020).

### SUMMARY

It would be desirable to generate a 3D model of the neuromuscular junction (NMJ) that can be used to study mechanisms of specific neuromuscular disorders and that is more appropriate for such studies than neuromuscular organoids known from prior art.

Aspects of the solution address this need. To be more precise, aspects of the solution relate to a complex 3D organoid model where all components of the NMJs are generated from the same progenitor population, self-organize and form functional NMJs.

To achieve this, the early embryonic developmental principles were transferred to a technical (in particular human) 3D in vitro model. A number of recent studies have established the common developmental origin of the spinal cord and associated musculoskeletal system from a bipotent axial stem cell population called neuromesodermal progenitors (NMPs) (Gouti et al., 2017; Henrique et al., 2015; Metzis et al., 2018; Tzouanacou et al., 2009; Wilson et al., 2009). NMPs reside in the node-streak border of elongating embryos and are important for axial elongation and formation of the spinal cord and musculoskeletal system (Cambray and Wilson, 2002; Henrique et al., 2015; Wilson et al., 2009). NMPs have been identified in the posterior epiblast of all vertebrate species examined, from fish to humans, indicating that they represent a conserved axial stem cell population during development (Kimelman, 2016; Olivera-Martinez et al., 2012). Yet, their potential to generate these tissues in an in vitro 3D organoid system remains unexplored.

An aspect of the solution relates to a method for generating a three-dimensional neuromuscular organoid in vitro. This method comprises the steps explained in the following.

First, a first cell culture comprising disease-specific neuromesodermal progenitor (NMP) cells is provided. The term “disease-specific neuromesodermal progenitor cells” refers to neuromesodermal progenitor cells that differ from healthy neuromesodermal progenitor cells by carrying genetic information of a patient suffering from a genetic disease. Thus, disease-specific neuromesodermal progenitor cells are neuromesodermal progenitor cells having or carrying a genetic defect. To give an example, it is possible to use neuromesodermal progenitor cells generated from patient-derived induced pluripotent stem cells (iPSC) originating from a patient having spinal muscular atrophy (SMA) or amyotrophic lateral sclerosis (ALS) or any other disease that affects the neuromuscular system. Such neuromesodermal progenitor cells are disease-specific neuromesodermal progenitor cells. They can also be denoted as iPSC-derived NMPs, wherein the iPSCs are cells originating from patients with a genetic disease that causes ALS, SMA, muscular dystrophies or any other disease that affects the neuromuscular system.

30% to 90% of the disease-specific neuromesodermal progenitor cells co-express BRACHYURY/S0X2 and 10% to 70% of the disease-specific neuromesodermal progenitor cells co-express TBX6. It surprisingly turned out that this specific expression pattern is a prerequisite for allowing an efficient generation of a neuromuscular organoid from disease-specific neuromesodermal progenitor cells, whereas such ratio of these expression markers is less important in case of healthy neuromesodermal progenitor cells.

The disease-specific neuromesodermal progenitor cells are cultivated in a first differentiation medium. This cultivation can be done under standard cultivation conditions such as a CO₂ content of 1% to 10% (v/v), in particular 2% to 9%, in particular 3% to 8%, in particular 4% to 7%, in particular 5% to 6%, and a temperature of 25° C. to 40° C., in particular 30° C. to 39° C., in particular 35° C. to 38° C., in particular 36° C. to 37.5° C., in particular 36.5° C. to 37° C. A CO2 content of approximately 5% and a temperature of approximately 37° C. are particular appropriate cultivation conditions.

The first differentiation medium can be a non-supplemented serum-free cell culture medium.

The term “non-supplemented” means in this context that the cell culture medium does not contain an inhibitor of Rho-associated, coiled coil containing protein kinase (ROCK) nor an activator of a growth factor signaling pathway nor an activator of insulin signaling pathway. However, the non-supplemented serum-free cell culture medium can contain components which are sometimes referred to as “supplements”, such as nutrients, amino acids, proteins and the like. Thus, the term “non-supplemented serum-free cell culture medium” does not necessarily refer to a bare cell culture medium not containing any additional supplemental components.

The first differentiation medium can, alternatively, be a serum-free cell culture medium supplemented with a ROCK inhibitor, an activator of a growth factor signaling pathway and/or an activator of insulin signaling pathway. In an embodiment, the serum-free cell culture medium is supplemented with a ROCK inhibitor and optionally supplemented with at least one activator of a growth factor signaling pathway and/or at least one activator of insulin signaling pathway.

Within a time period of 1 to 4 days, in particular 1 to 3 days, after cultivation start, the first differentiation medium is changed against a second differentiation medium. In this context, the second differentiation medium is either a non-supplemented serum-free cell culture medium or a serum-free cell culture medium supplemented with an activator of a growth factor signaling pathway and/or an activator of insulin signaling pathway. Thus, whereas the first differentiation medium can be a serum-free cell culture medium comprising a ROCK inhibitor, the second differentiation medium may not comprise such a ROCK inhibitor.

Within a time period of 1 to 4 days, in particular 1 to 3 days, after the first medium change, the second differentiation medium is changed against a non-supplemented serum-free cell culture medium. Thus, the types of possible supplements that can be added to the media are stepwise reduced.

After the second medium change, a three-dimensional neuromuscular organoid can be obtained from the non-supplemented serum-free cell culture medium. This can be done immediately after the latest medium change. Alternatively, it is also possible to further cultivate the neuromuscular organoid in the non-supplemented serum-free cell culture medium for an extended period of time (ranging, e.g., from a few days to a couple of weeks or even a couple of months up to more than one year).

The neuromuscular organoid is the first complex organoid model comprising two different tissues, namely spinal cord tissue and skeletal muscle tissue, that have co-developed and self-organized into an organoid.

To achieve the generation of disease-specific neuromesodermal progenitor cells in a particular appropriate way, the timing of treatment with the first differentiation medium is adjusted with respect to the timing applied in case of NMPs originating from a healthy individual (“healthy NMPs”). To be more precise, the timing of treatment ranges, in this embodiment, from 48 hours to 96 hours, in particular from 54 hours to 90 hours, in particular from 60 hours to 84 hours, in particular from 66 hours to 78 hours, in particular from 48 to 72 hours. The timing of treatment may be chosen depending on the line of the disease-specific neuromesodermal progenitor cells. Alternatively or additionally, the concentration of initial iPSCs plated ranges from 50.000/cm² to 100.000/cm², in particular from 60.000/cm² to 90.000/cm², in particular from 70.000/cm² to 80.000/cm².

In an embodiment, the second differentiation medium is a serum-free cell culture medium supplemented with at least one of an activator of a growth factor signaling pathway, and an activator of an insulin signaling pathway.

The disease-specific neuromesodermal progenitor cells can have a human or an animal origin. Thus, it is possible to generate human neuromuscular organoids or animal neuromuscular organoids. While it is generally possible to use either healthy or disease-specific neuromesodermal progenitor cells for manufacturing the neuromuscular organoid, the presently claimed solution focuses on disease-specific neuromesodermal progenitor cells (sometimes also referred to as diseased neuromesodermal progenitor cells. Consequently, the obtained neuromuscular organoid can be used in a particular appropriate way for studying specific neuronal or neuromuscular or muscular diseases in a non-healthy environment.

The developed 3D neuromuscular organoid (NMO) comprises spinal cord neurons, skeletal muscle cells and Schwann cells that are generated simultaneously and self-organize to form functional neuromuscular junctions. The NMO allow studying all the cells involved in neuromuscular junction formation and provides a patient-specific neuromuscular junction model (e.g., derived from (induced) pluripotent stem cells of the respective patient) to study specific diseases and perform drug screening studies.

In an embodiment, the disease-specific neuromesodermal progenitor cells are manufactured as described in Gouti et al., 2014. The content of this publication is herewith incorporated by reference in its entirety, in particular with respect to its content relating to the manufacturing of disease-specific neuromesodermal progenitor cells such as cultivation techniques, media and/or protocols.

In an embodiment, the non-supplemented serum-free cell culture medium is Dulbecco's Modified Eagle Medium (DMEM) comprising Ham's F12 medium (and optionally also comprising N2 medium), Advanced Dulbecco's Modified Eagle Medium (DMEM) comprising Ham's F12 medium (and optionally also comprising N2 medium) and/or neurobasal medium (optionally also comprising B27 medium) and/or neurobasal plus medium (optionally also comprising B27 medium). The non-supplemented serum-free cell culture medium can contain—as explained above—further components such as nutrients like proteins, amino acids, vitamins etc. N2 and B27 are examples of such further components. They are well known to a person skilled in the art and are described in detail by Price and Brewer (2001). Such a non-supplemented serum-free cell culture medium can also serve as a base medium for the serum-free cell culture medium supplemented with a ROCK inhibitor and/or at least one activator of a growth factor signaling pathway and/or an activator of insulin signaling pathway to be used in the precedingly explained step of the method disclosed and claimed herein.

In an embodiment, the non-supplemented serum-free cell culture medium comprises Dulbecco's Modified Eagle Medium (DMEM) comprising Ham's F12 medium or Advanced Dulbecco's Modified Eagle Medium (DMEM) comprising Ham's F12 medium additionally containing 1×N2 as well as neurobasal medium further containing 1×B27, L-glutamine, bovine serum albumin (BSA) fraction V, and 2-mercaptoethanol.

In an embodiment, the L-glutamine is present in a concentration of from 0.1 mM to 10 mM, in particular from 0.5 mM to 9 mM, in particular from 1 mM to 8 mM, in particular from 2 mM to 7 mM, in particular from 3 mM to 6 mM, in particular from 4 mM to 5 mM.

In an embodiment, the BSA fraction V is present in a concentration of from 1 μg/mL to 100 μg/ml, in particular from 10 μg/ml to 90 μg/mL, in particular from 20 μg/mL to 80 μg/mL, in particular from 30 μg/mL to 70 μg/mL, in particular from 40 μg/ml to 60 μg/mL, in particular from 40 μg/ml to 50 μg/mL.

In an embodiment, the 2-mercaptoethanol is present in a concentration of from 0.01 mM to 1 mM, in particular of from 0.05 mM to 0.5 mM, in particular from 0.1 mM to 0.3 mM.

In an embodiment, the ROCK inhibitor is present in a concentration of from 1 μM to 1000 μM, in particular from 10 μM to 500 μM, in particular from 20 μM to 250 μM, in particular from 30 μM to 100 μM, in particular from 40 μM to 90 μM, in particular from 50 μM to 80 μM, in particular from 60 μM to 70 μM.

In an embodiment, the ROCK inhibitor is (R)-(+)-trans-4-(1-Aminoethyl)-N-(4-Pyridyl)cyclohexanecarboxamide (Y-27632), in particular the dihydrochloride salt of this compound.

In an embodiment, the activator of a growth factor signaling pathway can be an activator of the fibroblast growth factor signaling pathway and/or an activator of the hepatocyte growth factor signaling pathway and/or an activator of the insulin-like growth factor signaling pathway and/or an activator of the epidermal growth factor signaling pathway and/or an activator of the nerve growth factor pathway and/or an activator of the platelet-derived growth factor signaling pathway.

A particular appropriate activator of the fibroblast growth factor signaling pathway is a fibroblast growth factor such as basic fibroblast growth factor 2 (FGF2) or another FGF isoform like FGF8, FGF17, FGF18 etc. These fibroblast growth factors activate the fibroblast growth factor signaling. In an embodiment, the fibroblast growth factor is used in a concentration lying in a range from about 1 to 1000 ng/mL, in particular 10 to 900 ng/mL, in particular 20 to 800 ng/mL, in particular 30 to 700 ng/mL, in particular 40 to 600 ng/mL, in particular 50 to 500 ng/mL, in particular 60 to 400 ng/mL, in particular 70 to 300 ng/mL, in particular 80 to 200 ng/mL, in particular 90 to 100 ng/mL.

A particular appropriate activator of the hepatocyte growth factor signaling pathway is a hepatocyte growth factor. In an embodiment, the hepatocyte growth factor is used in a concentration lying in a range from about 0.1 to 100 ng/mL, in particular 1 to 90 ng/mL, in particular 2 to 80 ng/mL, in particular 3 to 70 ng/mL, in particular 4 to 60 ng/mL, in particular 5 to 50 ng/mL, in particular 6 to 40 ng/mL, in particular 7 to 30 ng/mL, in particular 8 to 20 ng/mL, in particular 9 to 10 ng/mL.

A particular appropriate activator of the insulin-like growth factor signaling pathway is an insulin-like growth factor. In an embodiment, the insulin-like growth factor is used in a concentration lying in a range from about 0.1 to 100 ng/mL, in particular 1 to 90 ng/mL, in particular 2 to 80 ng/mL, in particular 3 to 70 ng/mL, in particular 4 to 60 ng/mL, in particular 5 to 50 ng/mL, in particular 6 to 40 ng/mL, in particular 7 to 30 ng/mL, in particular 8 to 20 ng/mL, in particular 9 to 10 ng/mL.

A particular appropriate activator of the epidermal growth factor signaling pathway is an epidermal growth factor. In an embodiment, the epidermal growth factor is used in a concentration lying in a range from about 0.1 to 100 ng/mL, in particular 1 to 90 ng/mL, in particular 2 to 80 ng/mL, in particular 3 to 70 ng/mL, in particular 4 to 60 ng/mL, in particular 5 to 50 ng/mL, in particular 6 to 40 ng/mL, in particular 7 to 30 ng/mL, in particular 8 to 20 ng/mL, in particular 9 to 10 ng/mL.

A particular appropriate activator of the nerve growth factor signaling pathway is a nerve growth factor. In an embodiment, the nerve growth factor is used in a concentration lying in a range from about 0.1 to 100 ng/mL, in particular 1 to 90 ng/mL, in particular 2 to 80 ng/mL, in particular 3 to 70 ng/mL, in particular 4 to 60 ng/mL, in particular 5 to 50 ng/mL, in particular 6 to 40 ng/mL, in particular 7 to 30 ng/mL, in particular 8 to 20 ng/mL, in particular 9 to 10 ng/mL.

A particular appropriate activator of the platelet-derived growth factor signaling pathway is a platelet-derived growth factor. In an embodiment, the platelet-derived growth factor is used in a concentration lying in a range from about 0.1 to 100 ng/mL, in particular 1 to 90 ng/mL, in particular 2 to 80 ng/mL, in particular 3 to 70 ng/mL, in particular 4 to 60 ng/mL, in particular 5 to 50 ng/mL, in particular 6 to 40 ng/mL, in particular 7 to 30 ng/mL, in particular 8 to 20 ng/mL, in particular 9 to 10 ng/mL.

In an embodiment, the first differentiation medium is a serum-free cell culture medium supplemented with a ROCK inhibitor, a fibroblast growth factor, an insulin-like growth factor and a hepatocyte growth factor.

In an embodiment, the second differentiation medium is a serum-free cell culture medium supplemented with an insulin-like growth factor and a hepatocyte growth factor. These growth factors have a particular appropriate influence on the growth and differentiation of neuromesodermal progenitor cells and thus on the generation of the neuromuscular organoid.

In an embodiment, the embodiments of the preceding paragraphs are combined.

In an embodiment, the non-supplemented serum-free cell culture medium is changed after having changed the second differentiation medium against the non-supplemented serum-free cell culture medium every 1 to 3 days, in particular every 1 to 2 days during a period of 10 days after cultivation start and every 2 to 5 days, in particular every 3 to 4 days, during a period exceeding 10 days after cultivation start. Such medium change will help maintaining the generated neuromuscular organoids in a vital state over an extended period of time.

In an embodiment, the method is carried out under agitation. This can, e.g., be achieved by a shaker such as an orbital shaker or by a bioreactor. In an embodiment, the agitation or rotation speed lies in a range of from 25 rounds per minute (rpm) to 200 rpm, in particular from 50 rpm to 150 rpm, in particular from 75 rpm to 100 rpm. Such a speed turned out to be particularly appropriate for a uniform growth of the organoids and a suited penetration of nutrients into the center of the organoids.

As explained above, the provided first cell culture comprises disease-specific neuromesodermal progenitor cells having a specific expression pattern with respect to the genes BRACHYURY/SOX2 and TBX6. In an embodiment, the provided first cell culture comprises 35% to 85%, in particular 40% to 80%, in particular 45% to 75%, in particular 50% to 70%, in particular 55% to 65%, in particular 60% to 65% of the disease-specific neuromesodermal progenitor cells that are co-expressing BRACHYURY/SOX2. At the same time, the first cell culture comprises in this embodiment 15% to 65%, in particular 20% to 60%, in particular 25% to 55%, in particular 30% to 50%, in particular 35% to 45%, in particular 35% to 40% of the disease-specific neuromesodermal progenitor cells that are co-expressing TBX6.

In an embodiment, the disease-specific neuromesodermal progenitor cells are derived from (diseased) pluripotent stem cells. Thus, in this embodiment, the disease-specific neuromesodermal progenitor cells are obtained by providing a second cell culture comprising pluripotent stem cells prior to performing the steps explained in the preceding paragraphs. The pluripotent stem cells contained in this second cell culture are cultivated in a first cultivation medium comprising a serum-free cell culture medium supplemented with a ROCK inhibitor and/or an activator of β-catenin signaling pathway with or without an activator of a growth factor signaling pathway.

In this context, the term “pluripotent stem cells” encompasses pluripotent stem cells originating from an embryo as well as induced pluripotent stem cells originating from a somatic cell (such as a skin cell, a fibroblast, a blood cell or any other somatic cell). Pluripotent stem cells are generally available from a stem cell culture that has been established without destroying the donor embryo. The pluripotent stem cells can be healthy pluripotent stem cells, i.e., originating from a healthy organism, or diseased pluripotent stem cells, i.e., originating from an organism suffering from a disease.

In an embodiment, the first cultivation medium comprises a serum-free cell culture medium supplemented at least with an activator of the β-catenin signaling pathway. Such an activator is particularly helpful in generating disease-specific neuromesodermal progenitor cells from pluripotent stem cells.

In an embodiment, the first cultivation medium comprises a serum-free cell culture medium supplemented at least with an activator of the β-catenin signaling pathway and/or an activator of a growth factor signaling pathway. Supplementing the serum-free cell culture medium with an activator of the β-catenin signaling pathway without an activator of a growth factor signaling pathway is particularly appropriate.

In an embodiment, the activator of the β-catenin signaling pathway is an inhibitor of glycogen synthase kinase 3 (GSK-3) activity or an activator of the Wnt signaling pathway that upregulates an expression of β-catenin. If GSK-3 activity is reduced, β-catenin is also upregulated since GSK-3 serves for a degradation of β-catenin.

In an embodiment, the activator of the β-catenin signaling pathway is at least one of the following compounds: 6-[[2-[[4-(2,4-Dichlorophenyl)-5-(5-methyl-1H-imidazol-2-yl)-2-pyrimidinyl]amino]ethyl]amino]-3-pyridinecarbonitrile (CHIR99021 or 0T99021), N-6-[2-[[4-(2,4-Dichlorophenyl)-5-(1H-imidazol-1-yl)-2-pyrimidinyl]amino]ethyl]-3-nitro-2,6-pyridine-diamine (CHIR98014), BIO-acetoxime, dynein light intermediate chain 1 (LiC1), 3-[(3-Chloro-4-hydroxyphenyl)-amino]-4-(2-nitrophenyl)-1H-pyrrol-2,5-dione (SB415286), N-(4-M ethoxybenzyl)-N′-(5-nitro-1,3-thiazol-2-Aurea (AR-A014418), 1-azakenpaullone, and bis-7-indolyl maleimide.

In an embodiment, the activator of a growth factor signaling pathway can be an activator of the fibroblast growth factor signaling pathway and/or an activator of the hepatocyte growth factor signaling pathway and/or an activator of the insulin-like growth factor signaling pathway and/or an activator of the epidermal growth factor signaling pathway. Appropriate examples of such activators as well as appropriate concentrations are indicated above with respect to the first and second differentiation medium. This information can be directly transferred to the first cultivation medium.

In an embodiment, the activator of a growth factor signaling pathway is a fibroblast growth factor such as basic fibroblast growth factor 2 (FGF2) or another FGF isoform like FGF8, FGF17, FGF18 etc. These fibroblast growth factors activate the fibroblast growth factor signaling. In an embodiment, the fibroblast growth factor is used in a concentration lying in a range from about 1 to 1000 ng/mL, in particular 10 to 900 ng/mL, in particular 20 to 800 ng/mL, in particular 30 to 700 ng/mL, in particular 40 to 600 ng/mL, in particular 50 to 500 ng/mL, in particular 60 to 400 ng/mL, in particular 70 to 300 ng/mL, in particular 80 to 200 ng/mL, in particular 90 to 100 ng/mL.

In an embodiment, the first cultivation medium is replaced by a second cultivation medium 1 to 3 days after cultivation start. It is to be noted in this context that the cultivation start for the cultivation of pluripotent stem cells in order to obtain disease-specific neuromesodermal progenitor cells is a different time point than the cultivation start of the cultivation of disease-specific neuromesodermal progenitor cells to obtain neuromuscular organoids. In this context, the second cultivation medium comprises a serum-free cell culture medium supplemented with at least one of an activator of β-catenin signaling pathway and an activator of a growth factor signaling pathway (but not supplemented with a ROCK inhibitor).

In an embodiment, the second cultivation medium is identical to the first cultivation medium except that the first cultivation medium contains a ROCK inhibitor and the second cultivation medium does not contain a ROCK inhibitor. Thus, the same (basic) serum-free cell culture medium can be used for the first cultivation medium and the second cultivation medium, wherein the supplements added to the serum-free cell culture medium differ between the first cultivation medium and the second cultivation medium.

In an aspect, the solution relates to a neuromuscular organoid that can be obtained by carrying out a method according to the preceding explanations. Such a neuromuscular organoid exhibits the following features:
Two different tissues, namely, spinal cord neurons and skeletal muscle fibers, are generated in parallel, self-organize and functionally interact.
Different cell types, namely, muscle fibers, spinal neurons including motor neurons and interneurons, glia cells, terminal Schwann cells, cartilage/bone and even endothelial cells develop simultaneously from a disease-specific neuromesodermal progenitor population enabling the study of the sequence of events that lead to disease and the interaction of the different cell types.
High reproducibility demonstrated by histological/immunofluorescence and single cell transcriptomic analyses in the organoid.
Neuromuscular organoids form functional neuromuscular junctions supported by the presence of terminal Schwann cells.
Neuromuscular organoids from all of the different human pluripotent stem cell (hPSC) lines examined started contracting in 3D between days 30 and 50 concomitant with the accumulation of alpha-bungarotoxin (aBTX) clusters in the muscle fibers. Blocking the acetylcholine receptor with curare resulted in organoid relaxation, indicating the presence of functional neuromuscular junctions between motor neurons and skeletal muscles in 3D.
Neuromuscular organoid contains myelinated axons at late stages of development.
Muscle contraction is controlled by functional NMJs and central pattern generators (CPGs).
It is the first human artificial organoid model where CPG-like networks are forming in 3D.
Neuromuscular organoids also contain microglia.
Muscle stem cells (satellite cells) are formed together with the skeletal muscle indicating that the organoids are useful for muscle regeneration studies.
Neuromuscular organoid can be maintained in culture for long periods of time (more than 1 year) without deterioration, indicating that neuromuscular organoid is useful to study maturation events.
Neuromuscular organoid represents a system amenable to functional testing and genetic manipulation.
Neuromuscular organoids be used to generate a complete NMJ and CPGs from patient-derived induced pluripotent stem cells (iPSCs) to study disease-causing mutations or the effects of reagents that have an impact on diseases.

In an embodiment, the neuromuscular organoid originates from diseased cells (such as genetically diseased cells) and thus comprises diseased cells (such as cells having a genetic defect or mutation). In an embodiment, the neuromuscular organoid originates from healthy cells in which a genetic mutation has been introduced. Also in such a case, the neuromuscular organoid comprises diseased cells (namely, cells having a genetic mutation). Especially such a neuromuscular organoid comprising diseased cells has not yet been described in prior art.

Such a neuromuscular organoid (made from disease-specific neuromesodermal progenitor cells) is—although it originates from and contains natural cells—an artificial system that is smaller and less complex than comparable neuromuscular arrangements within human or animal body. In particular, the neuromuscular organoid comprises only one specific part of the spinal cord, but not all parts generally present in the spinal cord. Natural neuromuscular arrangements always comprise more cell types than the neuromuscular organoid manufactured according to the presently described method. Natural neuromuscular arrangements also contain a vascular system and thus show vascularization. In contrast, the neuromuscular organoid as described herein does not comprise, in an embodiment, such an organized vascular network and can thus be well distinguished from natural neuromuscular arrangements. Rather, the neuromuscular organoid comprises, in an embodiment, vascular cells present in a non-organized form.

In an aspect, the solution relates to a first method of studying the development and/or mechanism of a disease in vitro. This method comprises subjecting a neuromuscular organoid obtained or obtainable by a method as disclosed herein to a reagent (in particular to a pharmaceutically active reagent) and observing an effect of the reagent on the neuromuscular organoid. In doing so, it is possible to test different reagent with respect to their influence on healthy or diseased neuromuscular organoids, i.e., to perform a drug screening. Thus, the presently described neuromuscular organoid represents a universal testing platform for drugs and other reagents to be used in treating a disease that can affect a neuromuscular arrangement in vivo.

In an aspect, the solution relates to a second method of studying the development and/or mechanism of a disease in vitro. This method comprises comparing a first neuromuscular organoid with a second neuromuscular organoid. The first neuromuscular organoid is obtained or is obtainable according to the presently described method, wherein this first neuromuscular organoid is made from a first type of neuromesodermal progenitor cells. This first type of neuromesodermal progenitor cells has a first genetic constitution (gene pool). The first neuromuscular organoid (or its behavior, respectively) is compared with a second neuromuscular organoid that is also obtained or is obtainable according to a method according to the preceding explanations. However, the second neuromuscular organoid is made from a second type of neuromesodermal progenitor cells, wherein this second type of neuromesodermal progenitor cells has a second genetic constitution (gene pool). In this context, the second genetic constitution differs from the first genetic constitution. Thus, it is, e.g., possible to induce a specific mutation in the first genetic constitution of the first neuromesodermal progenitor cell. Then, the second neuromesodermal progenitor cell type having the second genetic constitution will result. Thus, it is possible to study genetic modifications and their influence on a disease affecting a neuromuscular arrangement such as the neuromuscular organoid.

In an embodiment, the disease to be studied by either of the precedingly explained methods is a motor-neuron disease, a neuromuscular disease, a rare neuromuscular disease, a disease affecting the central nervous system of a patient, a disease affecting the neuromuscular or muscular system of a patient, a myopathy, or an auto-immune neuromuscular disease. Non-limiting examples of such diseases are amyotrophic lateral sclerosis (ALS), spinal bulbar muscular atrophy (SBMA), spinal muscular atrophy (SMA), Charcot-Marie-Tooth disease (CMT), giant axonal neuropathy (GAN), congenital myasthenic syndromes (CMS), Lambert-Eaton myasthenic syndrome (LEMS), myasthenia gravis, Duchenne muscular dystrophy, Becker muscular dystrophy, Myotonic muscular dystrophy, congenital muscular dystrophy, distal muscular dystrophy, Limb-girdle muscular dystrophy, Emery-Dreifuss muscular dystrophy, mitochondrial myopathies, congenital or distal myopathy.

In an embodiment, the motor-neuron disease is amyotrophic lateral sclerosis or spinal muscular atrophy. In an embodiment, the myopathy is Becker's muscular dystrophy or Duchenne's muscular dystrophy. In an embodiment, the auto-immune neuromuscular disease is myasthenia gravis.

In an aspect, the solution relates to a kit that comprises components that are particularly appropriate to carry out the precedingly explained method for generating a neuromuscular organoid. Such a kit comprises a non-supplemented serum-free cell culture medium and at least one supplement. The serum-free cell culture medium comprises Dulbecco's Modified Eagle Medium (DMEM) comprising Ham's F12 medium additionally containing 1×N2 or Advanced Dulbecco's Modified Eagle Medium (DMEM) comprising Ham's F12 medium additionally containing 1×N2. The serum-free cell culture medium of the kit further comprises neurobasal medium further containing 1×B27, L-glutamine, bovine serum albumin (BSA) fraction V, and 2-mercaptoethanol. The supplement is a ROCK inhibitor and/or an activator of a growth factor signaling pathway and/or an activator of insulin signaling pathway.

In an embodiment, the serum-free cell culture medium comprises a mixture of the Dulbecco's Modified Eagle Medium (DMEM) comprising Ham's F12 medium or the Advanced Dulbecco's Modified Eagle Medium (DMEM) comprising Ham's F12 medium and the neurobasal medium in a ratio (volume by volume) of 1:3 to 3:1, in particular of 1:2 to 2:1, in particular of 1:1.5 to 1.5:1, in particular of around 1:1.

All embodiments of the method for generating a three-dimensional neuromuscular organoid in vitro can be combined in any desired way and can be transferred either individually or in any arbitrary combination to the described neuromuscular organoid, the methods of studying the development and/or mechanism of a disease and the kit. Furthermore, all embodiments of the described neuromuscular organoid can be combined in any desired way and can be transferred either individually or in any arbitrary combination to the described method for generating a neuromuscular organoid, to the described methods of studying the development and/or mechanism of a disease and to the kit. Furthermore, all embodiments of the described methods of studying the development and/or mechanism of a disease can be combined in any desired way and can be transferred either individually or in any arbitrary combination to the described method for generating a three-dimensional neuromuscular organoid in vitro, to the described neuromuscular organoid, to the respective other method of studying a disease in vitro and to the described kit. Finally, all embodiments of the described kit can be combined in any desired way and can be transferred either individually or in any arbitrary combination to the described method for generating a three-dimensional neuromuscular organoid, to the described neuromuscular organoid, and to any of the described methods of studying the development and/or mechanism of a disease in vitro.

Further details of aspects of the solution will be explained with respect to exemplary embodiments and accompanying Figures.

### BRIEF DESCRIPTION OF THE DRAWINGS

FIG. 1A shows a schematic representation of the strategy used to generate NMOs from hPSCs.

FIG. 1B shows immunofluorescence analysis of sectioned organoids at day 5 showing the expression of the neural progenitor marker SOX1 and CDH6 in a polarized region of the organoid. PAX3⁺ cells are located in the SOX1⁺ region but also in the SOX1⁻ mesodermal region. Arrows indicate the neural (N) and mesodermal (M) region of the organoid. The posterior identity of the organoid is determined by the expression of HOXC9 and HOXC10.

FIG. 1C shows the percentage of elongated organoids at day 5 of NMO development in different hPSC lines (H9, H9^{SOX2GFP}, XM001 and H1) (n=1145, N=21; “n” indicating here and in the following the number of organoids analyzed; “N” indicating here and in the following the number of experiments from each cell line).

FIG. 1D shows bright-field images of representative organoids at different stages (day 1, day 5, day 50 and day 100).

FIG. 1E shows a graph showing the growth (average diameter) of NMOs during different development stages.

FIG. 1F shows UMAP projection and determined clusters of day 5 organoids reveals four main populations.

FIG. 1G shows UMAP plots showing gene expression levels of representative signature genes for each cluster.

FIG. 1H shows dot plot showing expression of representative genes across the main four clusters. The size of each circle reflects the percentage of cells in a cluster where the gene is detected, and the color reflects the average expression level within each cluster (blue: low expression, red: high expression). Data are represented as mean±standard deviation (SD).

FIG. 2A shows a UMAP plot of integrated day 5 and day 50 datasets. The four main cell clusters at day 5 are colored whereas day 50 cells are all grey. Dots represent individual cells and colors indicate cluster identity.

FIG. 2B shows a UMAP plot of the day 50 NMO cells revealed 5 main cell clusters. Dots represent individual cells and colors indicate cluster identity.

FIG. 2C shows the velocity field overlaid with the UMAP plot indicating the developmental trajectories that day 5 and day 50 cells follow. Colors indicate the different cell cycle phases, G1 as orange color; S as green color and G2-M as blue color. The main developmental trajectories are shown with colored arrows; blue for the neural lineage and red for the skeletal muscle lineage. Density plot of day 5 (dark grey) and day 50 (light grey) single cells with colored arrows show the main developmental trajectories that single cells follow as they commit to specific lineages.

FIG. 2D shows a sub-clustering of the neural and skeletal muscle trajectories revealing 8 additional clusters. Shades of blue and red show the different neural and muscle sub-clusters respectively.

FIG. 2E-F show a dot plot showing expression of representative genes across the 3 sub-clusters present in the skeletal muscle trajectory (FIG. 2E) and across the 5 sub-clusters present in the neural differentiation trajectory (FIG. 2F). The size of each circle reflects the percentage of cells in a cluster where the gene is detected, and the color intensity reflects the average expression level within each cluster (blue: low expression, red: high expression).

FIG. 2G evaluates the reproducibility of gene expression patterns in organoids by single cell sequencing on four individual day 50 NMOs from two different batches of the H9 hPSC line. The UMAP analysis of each organoid shows that all the different populations are reproducibly generated.

FIG. 2H shows the percentage of cells from each individual organoid belonging to each cell sub-cluster in identically processed datasets from day 50 NMOs (two batches, n=4 individual organoids).

FIG. 3A shows an immunofluorescence analysis of NMO sections at day 50 indicating the presence of neurons (TUJ1+ cells) that project axons into the organized skeletal muscle region labelled with myosin skeletal fast (Fast MyHC).

FIG. 3B is a schematic representation of the neuromuscular organization in the human body where spinal cord neurons from the ventral horn connect with limb skeletal muscles to form functional NMJs and control movement.

FIG. 3C shows a percentage of neural and muscle cells indicating the reproducibility of NMO generation in at least three different organoids (n) from three different experimental batches (N) in the H9 line (n=12, N=3).

FIG. 3D-G shows transmission electron microscopy results revealing that day 70 organoids consist of two major functional compartments. NMO muscle region showing a high degree of maturation with sarcomere features such as parallel-aligned myosin-actin filaments, distinct Z-lines (Z) and M-bands (M), glycogen granules (Gly) and areas with grouped mitochondria (FIG. 3D). Higher magnification of a sarcomeric region is shown in FIG. 3E; NMO neuronal region showing densely packed parallel-aligned axons and intact synapses. The neuronal axons are rich in mitochondria (Mi) and neuro-filaments (FIG. 3F). In higher magnifications the synapses show the formation of synaptic clefts and the presence of synaptic vesicles (SV) at the active zone (asterisks) (FIG. 3G). Data are represented as mean±SD.

FIG. 4A shows a section of whole organoid at day 50 revealing the expression of the sarcomeric protein TITIN in the skeletal muscle fibers and the presence of cells that express the glial fibrillary acidic protein (GFAP).

FIG. 4B shows that the number of glia cells (GFAP+) in NMOs is significantly increased from day 50 (14.2%±4.1) to day 150 (58.2%±6.8), consistent with the later development of glia cells in vivo.

FIG. 4C shows a section of whole organoid at day 150 revealing the organized expression of the sarcomeric protein TITIN in the skeletal muscle fibers and the presence of cells that express the glial fibrillary acidic protein (GFAP).

FIG. 4D shows a higher magnification of the muscle region indicating the presence of muscle striations, revealed by TITIN immunofluorescence, and peripheral nucleation of the mature muscle fibers, revealed by DAPI. GFAP+ glia cells are also observed in close proximity to the muscle.

FIG. 4E shows that myelin basic protein (MBP) is detected at day 50 in NMO suggesting the presence of myelinated axons.

FIG. 4F shows a higher magnification of the neural region of the organoids indicating the presence of myelinated axons stained with MBP and TUJ1.

FIG. 5A shows that TUJ1⁺ neurites are in contact with α-bungarotoxin (αBTX) positive AChR clusters on fast-twitch skeletal muscle fibers (Fast MyHC⁺).

FIG. 5B shows a quantification of the number of αBTX clusters at day 50 NMO regions normalized for the number of Fast MyHC myofibers, in at least three organoids from three different experiments in three different cell lines (H1, H9 and XM001), indicating reproducible generation of NMJs among different experiments and lines (n=44, N=9). Quantification of αBTX⁺ clusters at regions of day 50, 100 and 150 organoid normalized for the number of Fast MyHC myofibers. The number of NMJs is significantly reduced between day 50 and day 100 NMOs whereas there is no significant difference between day 100 and day 150 NMOs (n=30, N=9).

FIG. 5C shows terminal Schwann cells (S100β⁺) capping the neurites in contact with αBTX clusters characteristic of functional NMJs. Higher magnification of a relevant region is shown.

FIG. 5D shows transmission electron microscopy pictures revealing that many axons are positioned close to muscle cells and some form NMJs. The muscle cell (MC) displays characteristic features such as a basal lamina (BL), protein rich densities and invaginations of the plasma membrane at the motor neuron-muscle contact site (SC: synaptic cleft). The neighboring MN axon shows pre-synaptic densities and synaptic vesicles close to the muscle cell (MC). Black and white arrowheads indicate pre- and post-synaptic densities respectively.

FIG. 5E shows graphs indicating displacement of a region of interest (ROI) in 250-seconds recordings of spontaneous contractions before and after administration of 10 uM Curare (a.u. arbitrary units).

FIG. 5F shows representative images of αBTX clusters in control and MG patient treated NMOs after three days of treatment with patient IgGs.

FIG. 5G shows quantification of αBTX clusters in control NMOs at day 50 and NMOs treated with auto-antibodies from MG Patient 1 and 2 respectively shows a significant reduction in the number of αBTX clusters in MG NMOs.

FIG. 5H-I show that the ratio of muscle contraction rate (FIG. 5H) and the amplitude of muscle contraction (FIG. 5I) are significantly reduced following a 3 day treatment with MG IgG autoantibodies but are not affected by treatment with IgG from a healthy subject. Data are represented as mean±SD.

FIG. 6A-B show an immunofluorescence analysis of PAX2/LHX1 expressed in spinal cord (V0, V1 and d16) interneurons and CHX10 expressed in V2a premotor interneurons revealing their presence in the neural part of day 50 NMOs.

FIG. 6C shows a quantification of PAX2⁺ (13.8%±5.5), PAX2⁺/LHX1⁺ (8.6%±2) and CHX10⁺ (9.3%±4) interneurons in NMOs at day 50 (N=3, n=9).

FIG. 6D shows calcium imaging at day 50 revealing spontaneous activity of the NMOs (n=5).

FIG. 6E shows a bright-field imaging of day 50 NMO in the multi-electrode array system.

FIG. 6F shows a plot indicating the average spike frequency before (spontaneous; sns) and after the application of 50 uM NMDA and 40 uM 5-HT in day 30 and day 50 NMOs. Day 50 NMOs show central pattern generator-like activity.

FIG. 6G shows a network graph indicating connectivity between electrodes recording spontaneous (left) or 50 uM NMDA/40 uM 5-HT at day 30 (middle) or 50 uM NMDA/40 uM 5-HT at day 50 stimulated electrical activity. Line thickness represents the number of times the paired firing occurred between electrodes, while node size shows the number of spikes captured by each single electrode during a recording. Stimulation of neurons at day 50 with NMDA/5-HT showed increased single firings, as well as an increase in paired electrode spikes. The position of the individual NMOs in the MEA grid is shown with a dashed line.

FIG. 7A shows a schematic diagram of the strategy used to study spinal muscular atrophy by manufactured neuromuscular organoids.

FIG. 7B shows NMPs derived from human induced pluripotent stem cells derived from a patient suffering from spinal muscular atrophy (SMA1).

FIG. 7C shows immunostaining for markers of neuromuscular junctions.

FIG. 8A shows a schematic diagram of the strategy used to study amyotrophic lateral sclerosis by manufactured neuromuscular organoids.

FIG. 8B shows NMOs developed from an ALS patient with mutation in the superoxide dismutase 1 (SOD1) gene.

FIG. 8C shows NMOs developed from an isogenic ALS patient line where the SOD1 gene mutation has been corrected.

FIG. 9A shows an example of two different NMOs that have been maintained for 300 days in culture.

FIG. 9B shows the presence of motor neurons after the long-term maintenance (300 days) of NMOs in culture.

### DETAILED DESCRIPTION

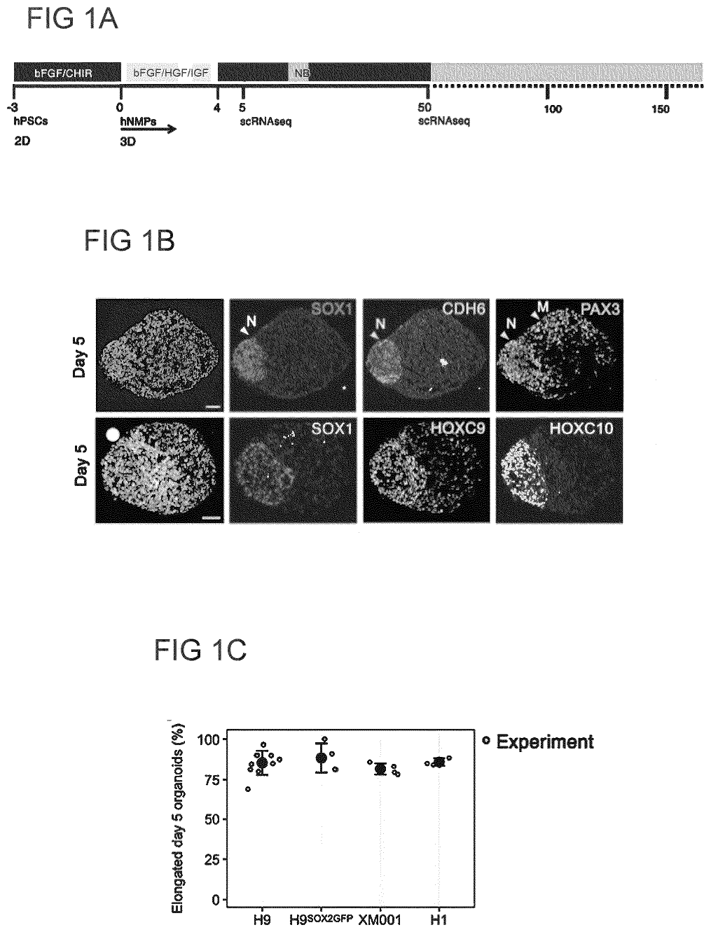

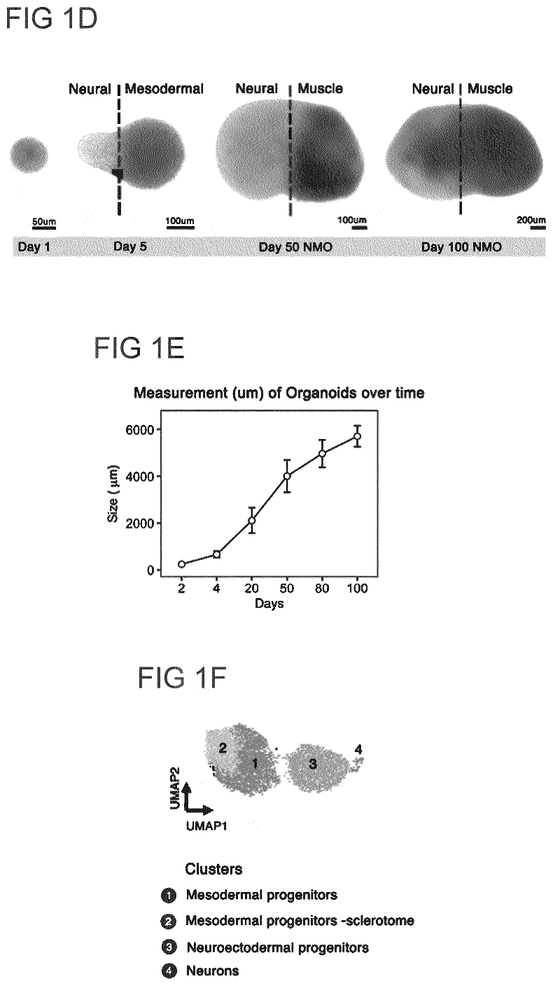

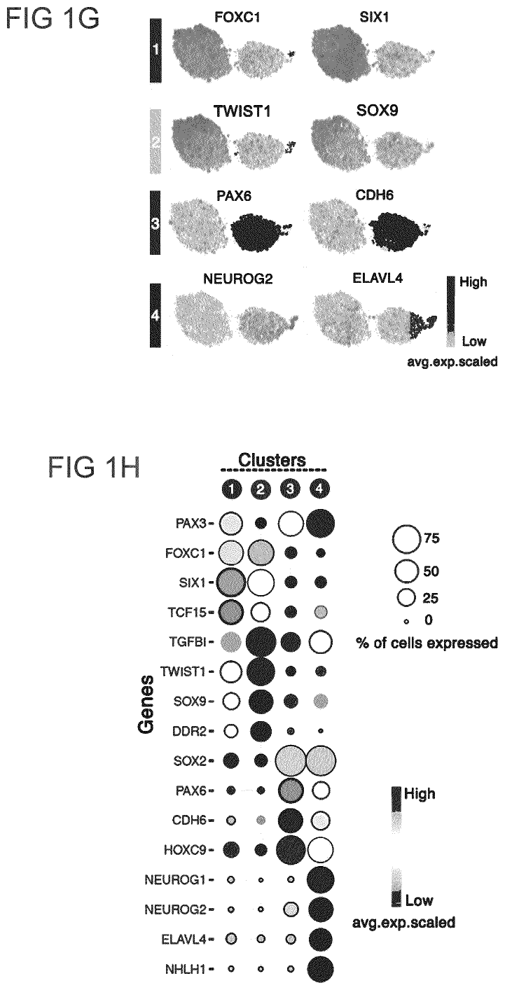

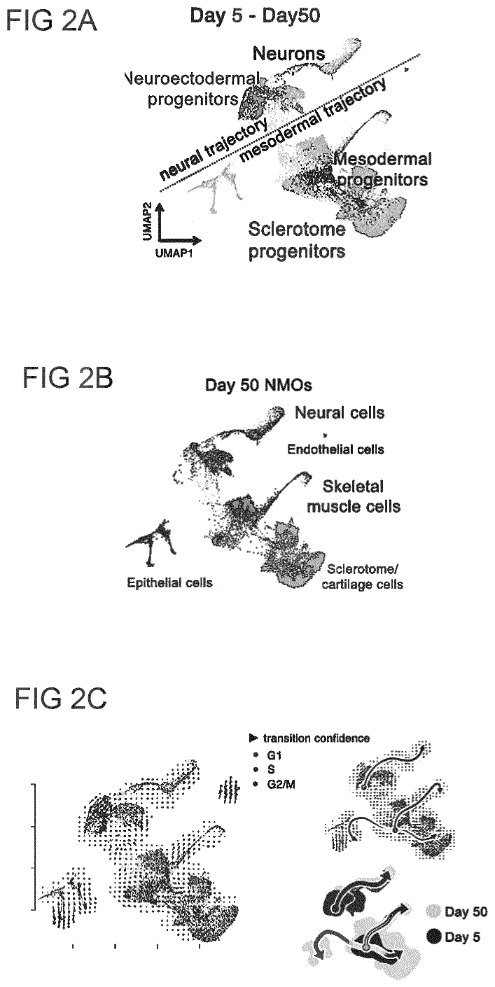

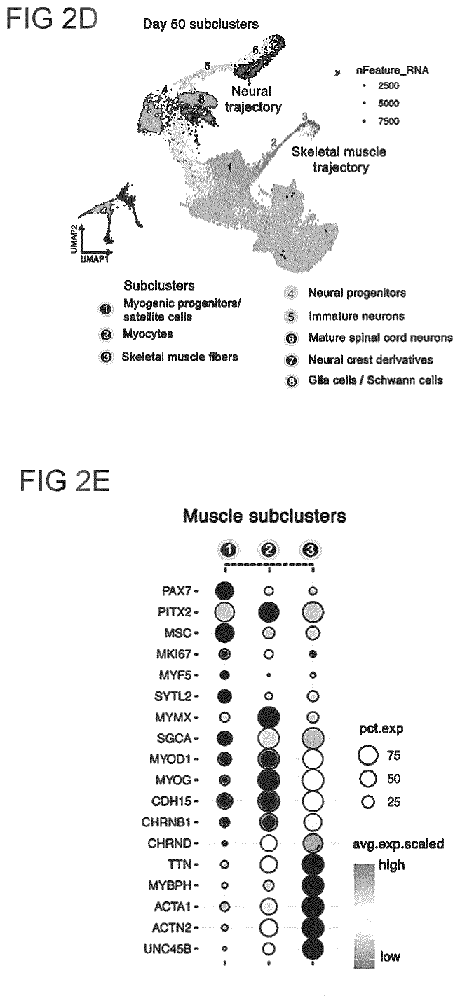

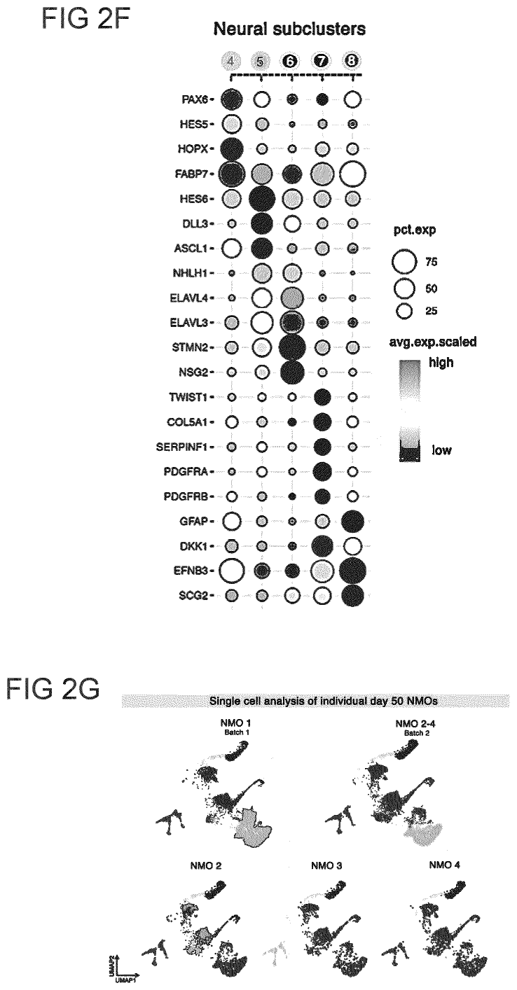

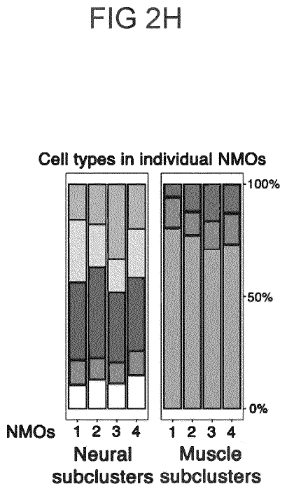

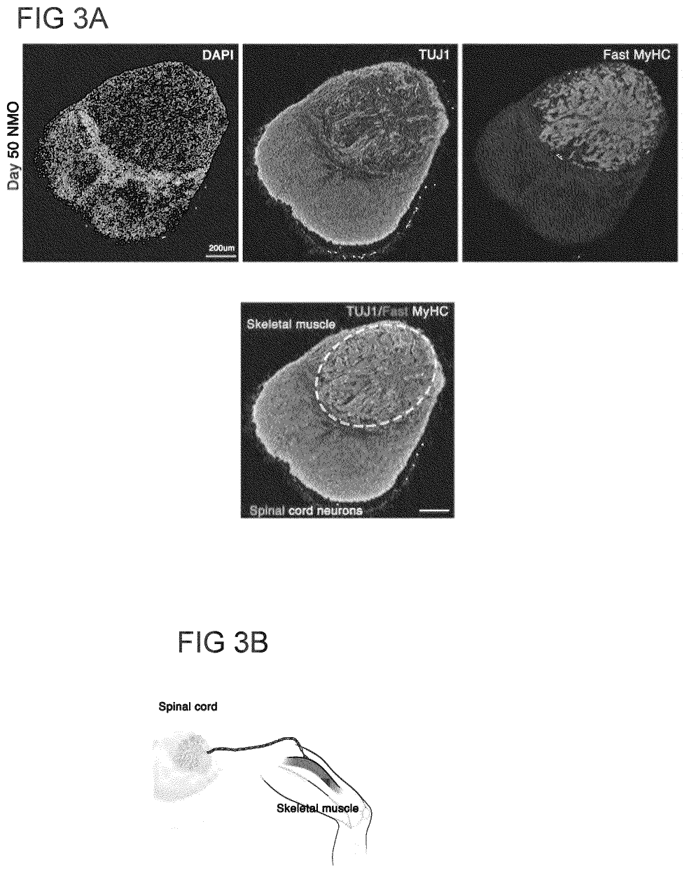

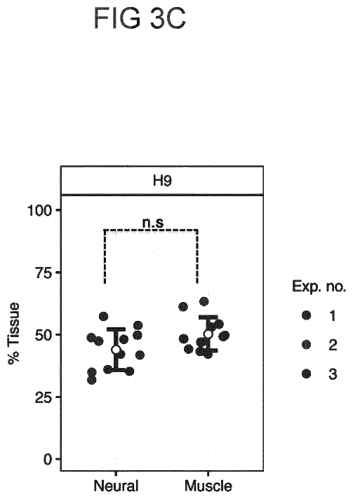

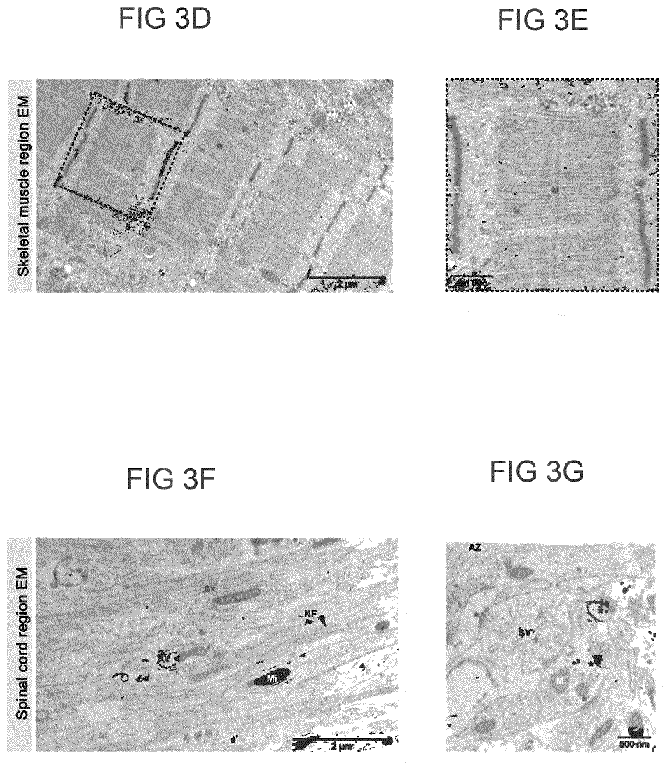

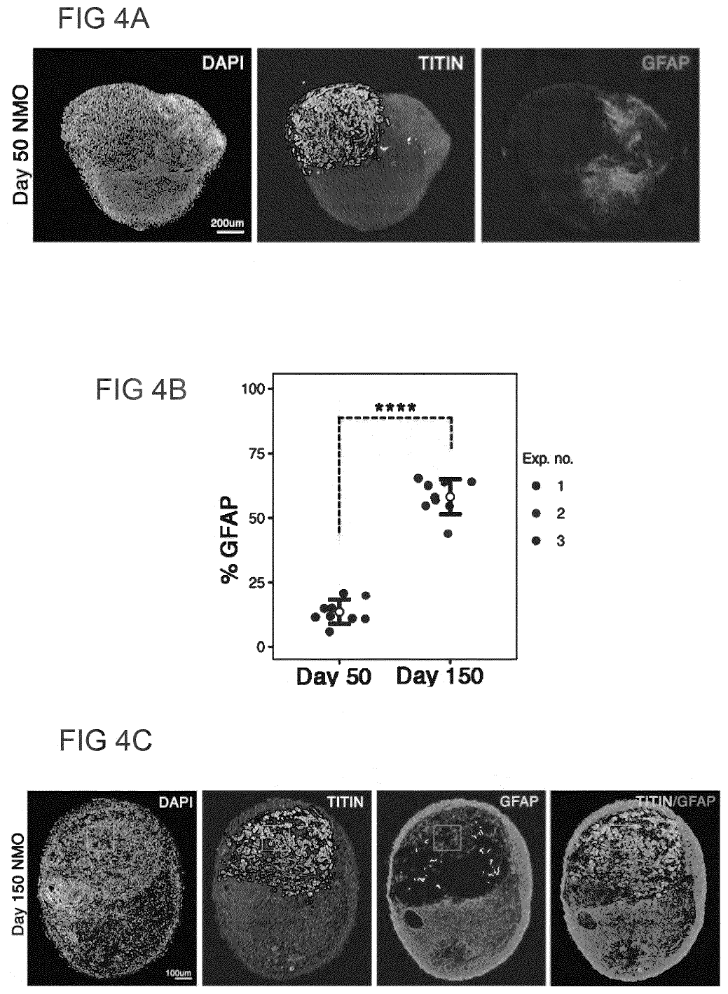

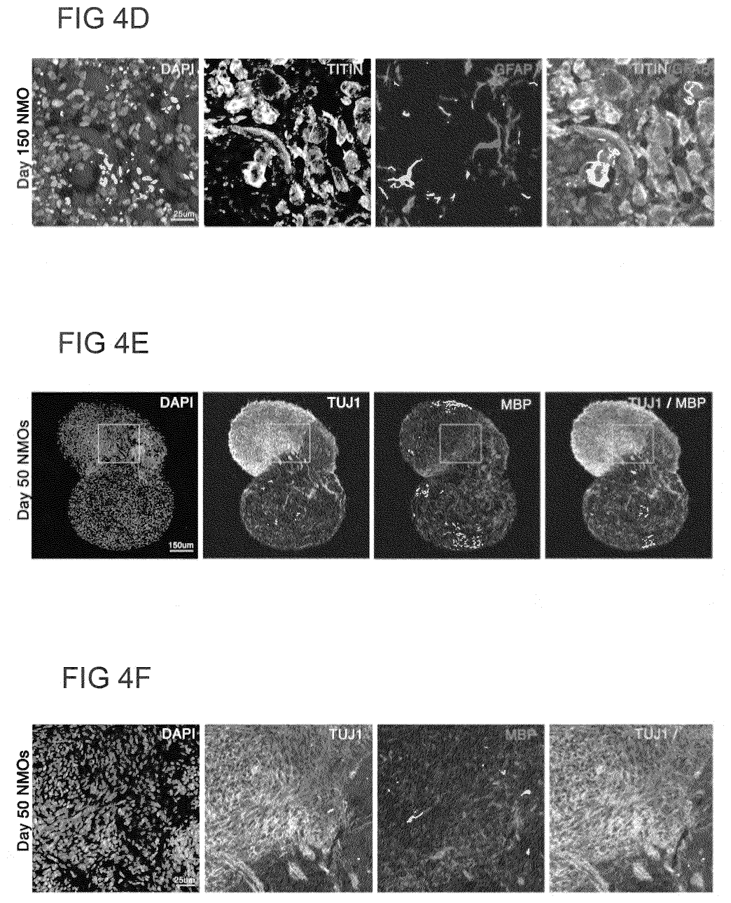

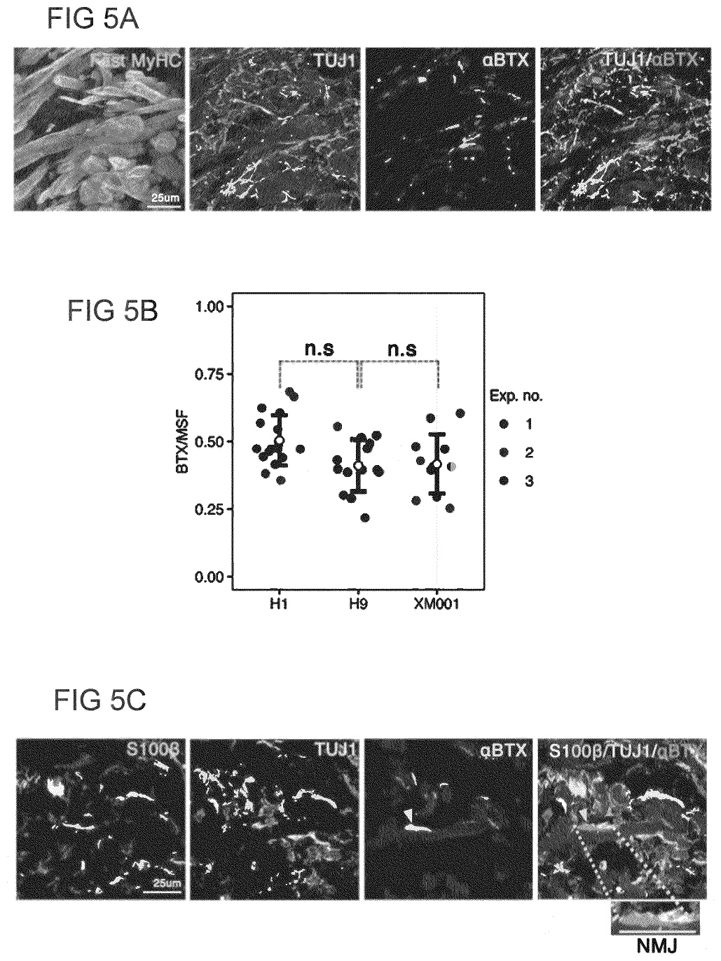

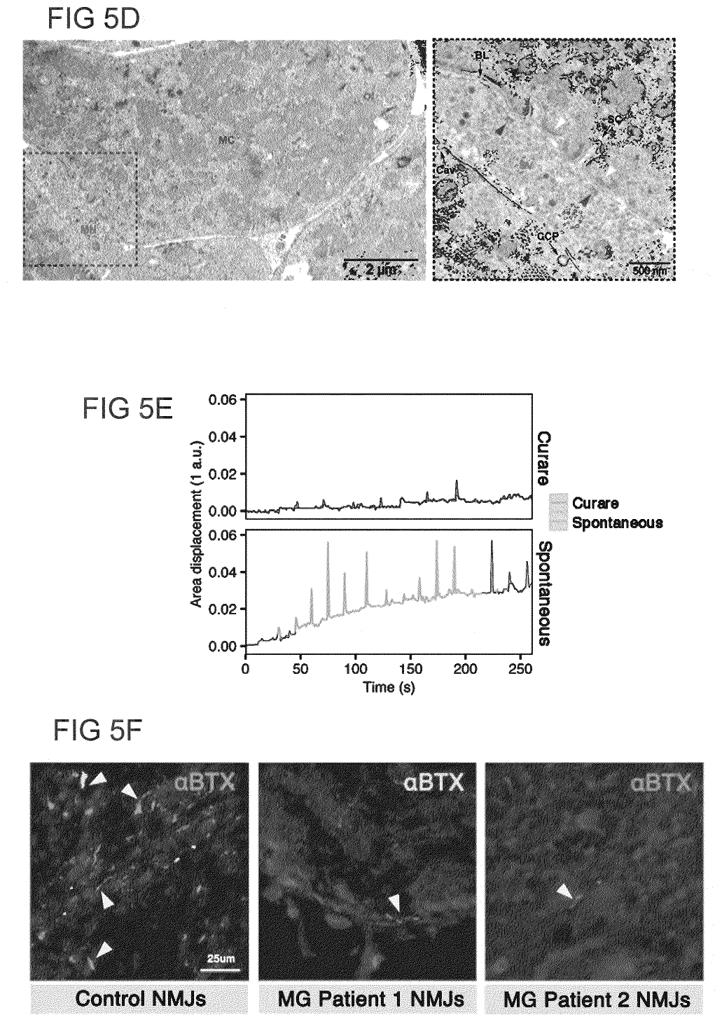

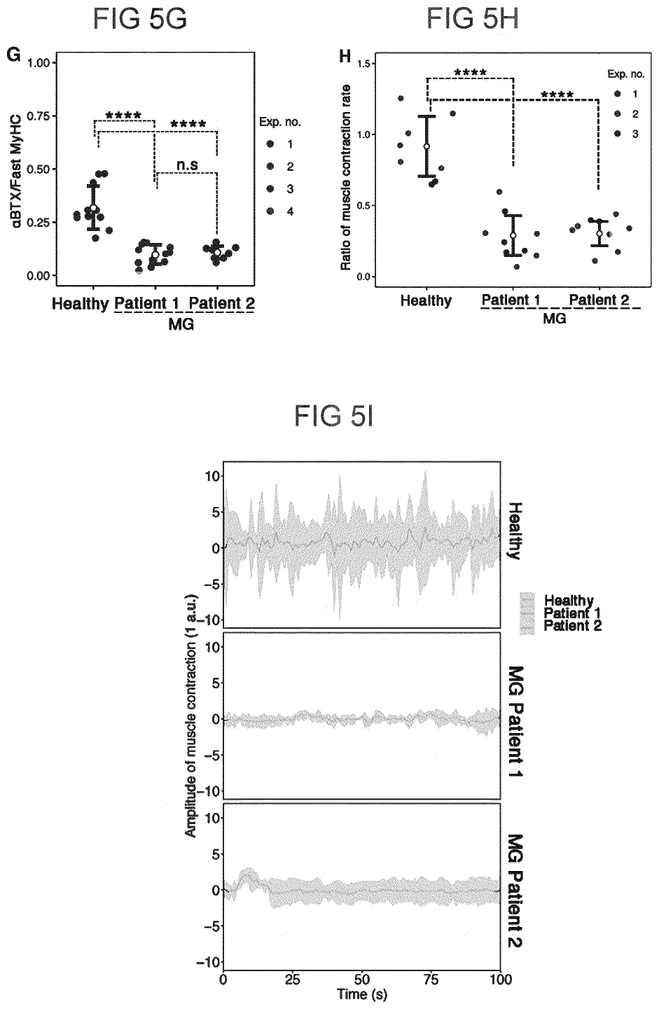

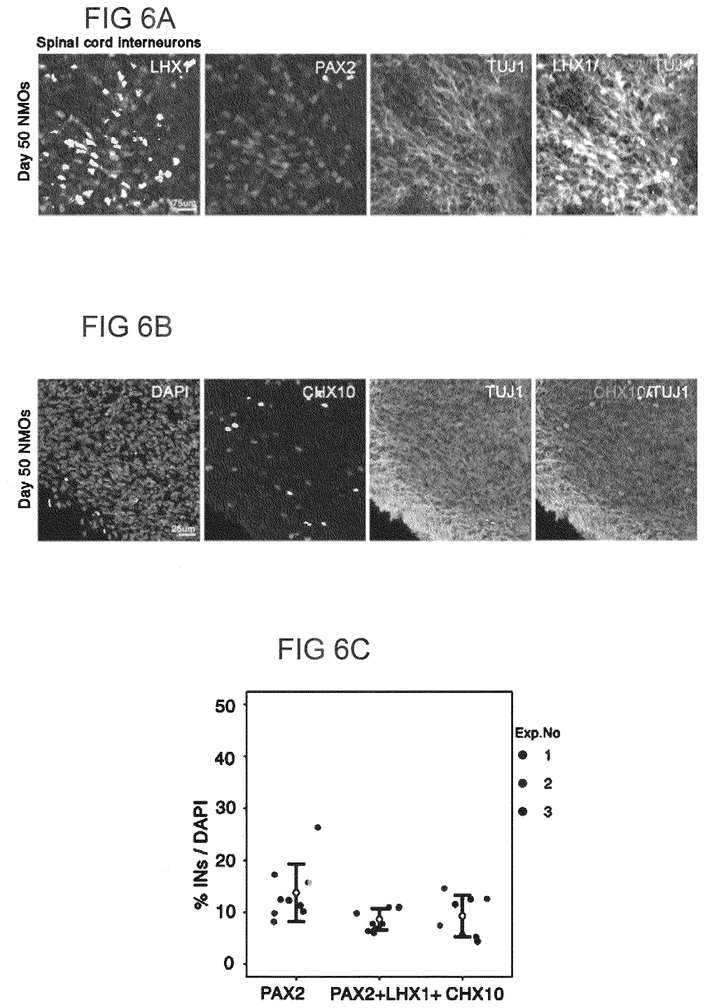

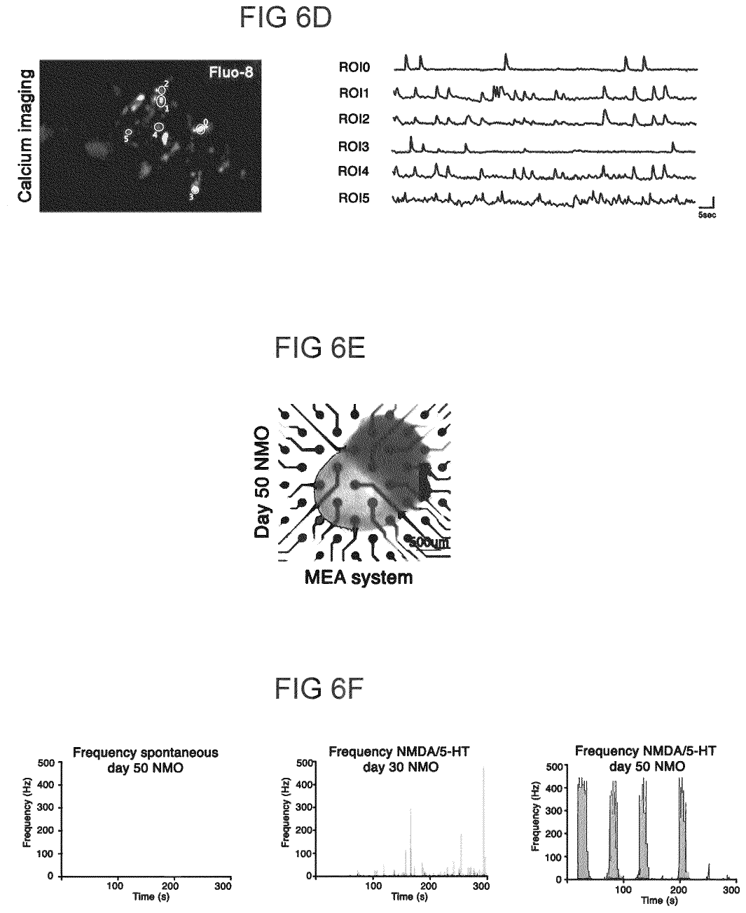

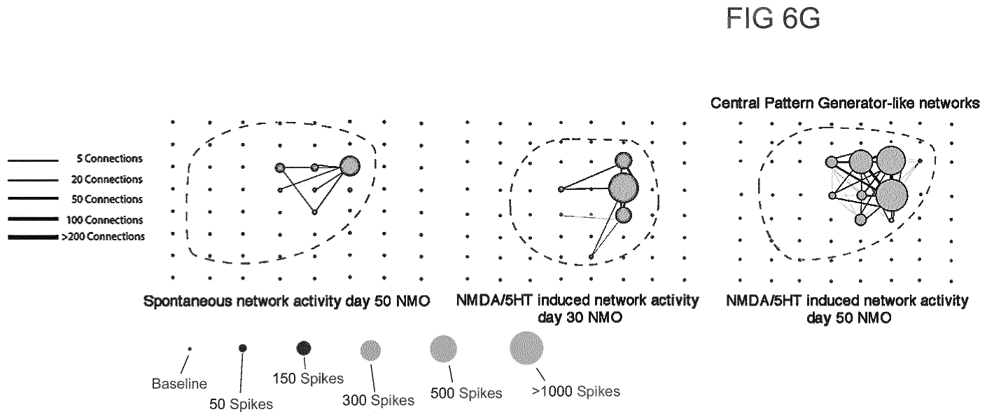

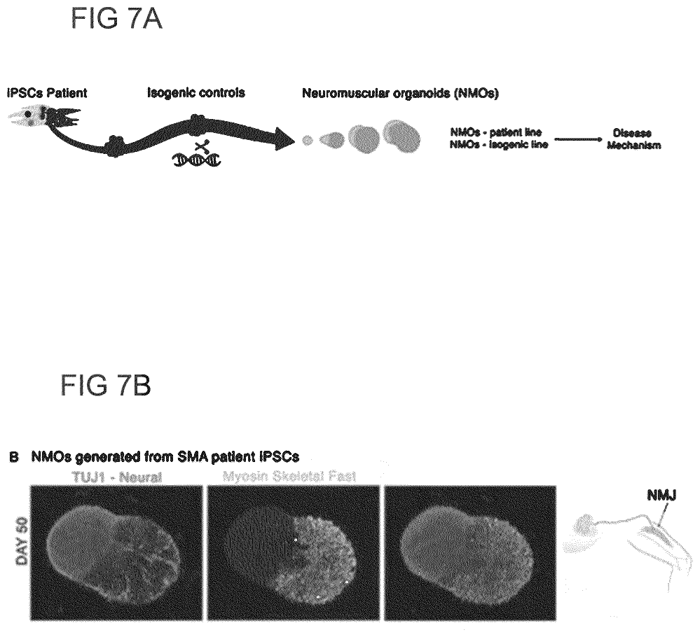

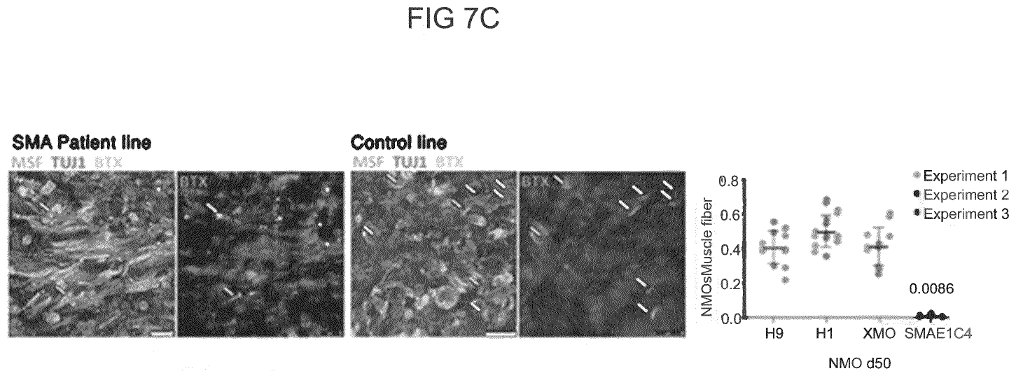

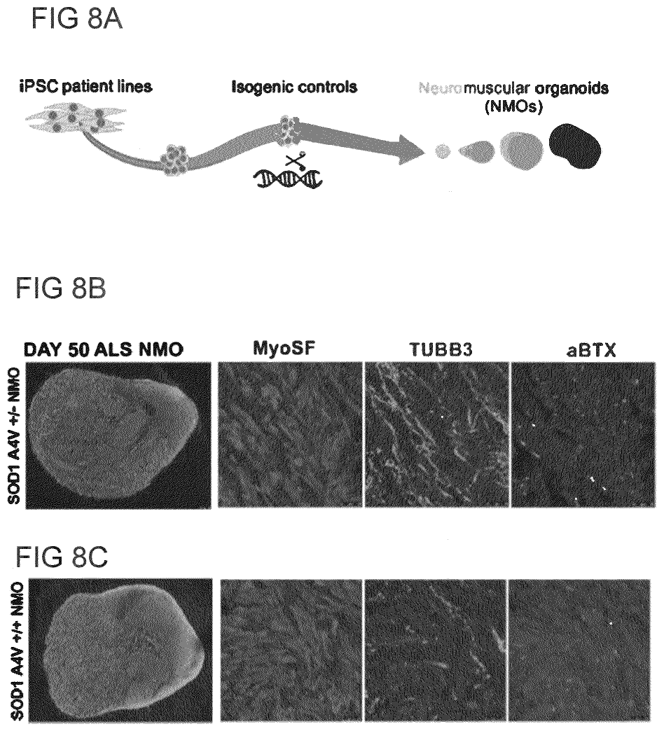

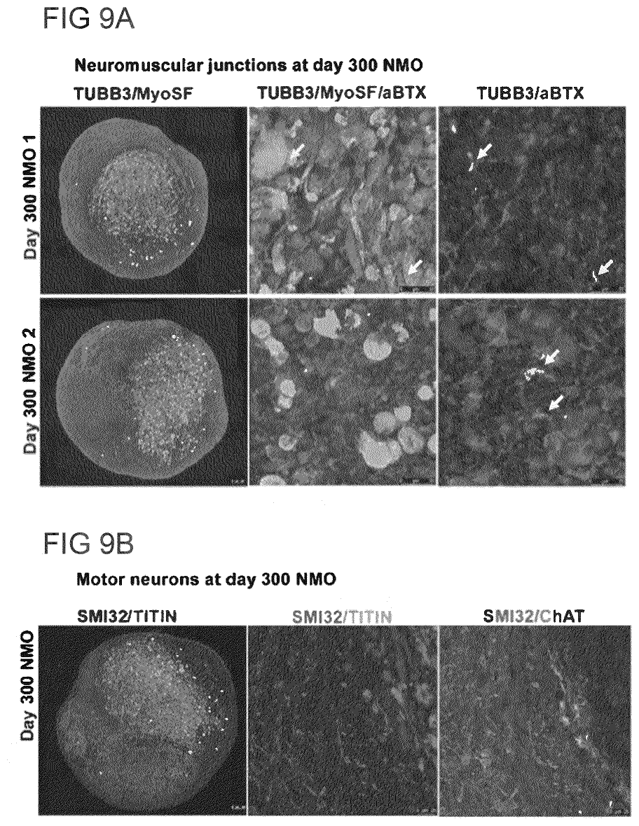

The Figures will be explained in the following in more detail with respect to exemplary embodiments. In brief, FIG. 1A-G relate to generating human self-organizing NMOs in 3D from NMPs. FIG. 2A-H show single cell analysis of day 5 to day 50 developmental trajectories and cellular composition of day 50 NMOs. FIG. 3A-G show the self-organization of spinal cord neurons and skeletal muscles in NMOs. FIG. 4A-F show glia development and NMO maturation between day 50 and day 150. FIG. 5A-I show the formation of functional NMJs at day 50 NMOs that enable modelling of Myasthenia Gravis. FIG. 6A-G show the formation of functional spinal cord networks with central pattern generator-like activity in NMOs. FIG. 7A-C relate to a disease model for studying spinal muscular atrophy with a neuromuscular organoid. FIG. 8A-C relate to a disease model for studying amyotrophic lateral sclerosis with a neuromuscular organoid. FIG. 9A-B relate to the long-term maintenance of NMOs in culture. NMOs were maintained for 300 days in culture without signs of deterioration as shown by the presence of spinal cord neurons, skeletal muscles and NMJs.

For the embodiments explained in the following, hPSC-derived NMPs were used, since they represent the building blocks of the posterior body (Frith et al., 2018; Gouti et al., 2017; Gouti et al., 2014; Verrier et al., 2018). In doing so, the simultaneous development of the spinal cord neural and mesodermal lineages was achieved in complex 3D organoids. The two lineages interact as they develop and, during their maturation, they self-organize to form functional NMJs comprising spinal cord neurons, skeletal muscles and terminal Schwann cells. The neuromuscular organoids, defined herein as NMOs, acquire a posterior axial identity, develop contractile activity driven by functional NMJs, are electrophysiologically active and form central pattern generator (CPG)-like circuits. The data shows that the NMO represents a complex model system, highly reproducible across experiments and different hPSC lines and amenable to functional testing and manipulation. They can be maintained as 3D structures for several months, while the component tissues mature, giving unprecedented access to human developmental events and allowing analysis of the specific contributions of different cell types to neuromuscular disorders. Finally, NMOs were used to model myasthenia gravis (MG), an autoimmune disorder that selectively targets the neuromuscular junction. Treatment of the NMOs with autoantibodies from myasthenia patient serum resulted in severe defects of the integrity of the NMJs and reduced contractile activity of the muscle, which represent key features of the disease pathology.

Self-Organization of hPSC Derived Neuromesodermal Progenitor Cells in 3D.

Human pluripotent stem cells (hPSCs) (H9, H1 human PSC lines and the XM001 iPSC line) were used to generate NMPs. NMPs are primed to develop exclusively into tissues of the posterior body (Deschamps and Duboule, 2017; Gouti et al., 2017; Metzis et al., 2018). Initial exposure of hPSCs to Wnt and FGF signals for three days resulted in the efficient generation of human NMP cells characterized by the co-expression of the neural progenitor marker SOX2, the nascent mesodermal marker Brachyury (T/BRA) and the posterior determinant CDX2 (Frith et al., 2018; Gouti et al., 2014; Verrier et al., 2018). NMP cells were then induced to form 3D aggregates on round-bottomed ultra-low-adhesion 96-well plates in neurobasal (NB) medium supplemented with growth factors (FGF/HGF/IGF) (FIG. 1A), which enhanced the expansion of mesodermal progenitors the first days of culture (Chal and Pourquie, 2017). Initially, the aggregates formed round structures, transiently containing NMP cells which underwent morphogenetic movements during days 0-5. The initial movement resulted in the segregation of a clear region corresponding to neuroectoderm, as evidenced by SOX1 and localized Cadherin-6 (CDH6) expression, and a mesodermal region expressing PAX3 but not SOX1 (FIG. 1B). The posterior identity was confirmed by strong expression of HOXC9 in the neural part of the developing organoid and HOXC10 expression in the polarized tip (FIG. 1B) (Deschamps and Duboule, 2017; Forlani et al., 2003). To assess the reproducibility of the system, comparing different organoids, experiments (batches) and different cell lines, the number of organoids that showed the characteristic elongated pattern at day 5 was quantified in four different hPSC lines (H9, H9^{SOX2GFP}, H1, XM001) and at least 3 different experiments. More than 80% of the organoids had the characteristic elongated morphology, independent of cell line, clone or batch (FIG. 1C-D). The organoids grew rapidly until day 50, when they reached a mean core size of 4-5 mm in diameter. Their growth continued but slowed, and they reached an average of 6 mm in diameter at day 100 (FIG. 1D-1E). Notably, these organoids can survive for prolonged periods (currently 1 year in culture) without obvious signs of deterioration.

To determine the major cell populations at the early stages of organoid formation and to assess their differentiation potential, the transcriptomes of 5,135 cells was analyzed from day 5 organoids (H9 line) using microdroplet-based single-cell RNA-sequencing. On average, 11,840 unique reads were determined, accounting for 3,333 genes, per cell. Application of a non-linear dimensionality reduction visualization algorithm, uniform manifold approximation and projection (Becht et al., 2018) to all single cells resulted in the identification of 4 clusters at day 5 (FIG. 1F). The main separation was between two mesodermal (cluster 1, 2) and two neural clusters (cluster 3, 4). Cells in cluster 1 and 2 were expressing genes such as FOXC1 and SIX1, characteristic of the developing paraxial mesoderm and somite; cluster 2 cells expressed additionally genes such as TGFBI, TWIST1 and SOX9 typical of the developing ventral somite and sclerotome (FIGS. 1G and 1H). Cells in cluster 3 were expressing genes associated with the neuroectodermal lineage such as PAX6, SOX2 and CDH6, whereas the few cells of cluster 4 were already committed to a more differentiated state expressing neural specific genes such as NEUROG1, NEUROG2, NHLH1 and ELAVL4 (FIG. 1G, 1H). In all four clusters, posterior HOX genes were up regulated while anterior genes such as FOXG1, OTX1, DLX2, DLX5, SIX3 were not detected, further confirming the posterior identity of these organoids. To further evaluate the reproducibility of the day 5 organoids at the cellular level, cell hashing was used to analyze two different organoid populations (Stoeckius et al., 2018). Indeed, the distribution of the cells and different clusters were strikingly similar in the two samples. Taken together, the analysis at day 5 revealed that the major populations, normally derived from NMPs in the developing embryo, were now generated from human NMPs in vitro, in 3D organoids (Gouti et al., 2017).

### Self-Organization of Spinal Cord Motor Neurons and Skeletal Muscles in NMOs

To examine the potential of these organoids to self-organize and generate spinal cord neurons and skeletal muscles, the organoids were maintained for prolonged periods of time in 3D using minimal media conditions (NB) and analyzed at different time points. Analysis at day 10 revealed the presence of OLIG2⁺ ventral spinal cord progenitor cells and co-expression of the trunk neural crest markers SOX9, SOX10 with HOXC9. At day 20, expression of OLIG2 was downregulated, concomitant with the differentiation of MN progenitors to numerous ISLET1⁺ MNs. On the other hand, the myogenic progenitor markers, MYOD/DESMIN were specifically expressed in the mesodermal part of the organoid. The presence of both MNs and myoblasts at that stage suggested that the organoids were maturing over time following the in vivo sequence of events.

At day 50, NMOs retained the characteristic morphology whereby the neural and muscle region could be clearly distinguished by bright field imaging (FIG. 1D). To examine the full repertoire of cells generated during organoid development, single-cell RNA-sequencing of four individual organoids was performed (total cells 17,294) at day 50. UMAP projection of day 5 and day 50 single cells from corresponding organoids revealed two major differentiation routes, one associated with a neural differentiation trajectory and one associated with a mesodermal differentiation trajectory (FIG. 2A, 2B). UMAP analysis of day 50 NMOs suggested the presence of 5 broad cell populations (FIG. 2B), one neural population (blue) and four mesodermal derived populations corresponding to skeletal muscle (red), epithelial (brown), endothelial (black) and sclerotome/cartilage (orange) cells (FIG. 2A-2B). Cell cycle analysis of single cells as they transit from day 5 to day 50 revealed that most day 5 cells were in the S and G2/M phases while day 50 cells were mostly in the G1 phase (FIG. 2C). These results were consistent with the ongoing maturation of the NMOs.

To identify distinct cell populations at day 50 NMOs, the focus was put on the skeletal muscle and neural specific trajectories. RNA velocity unveiled the dynamics and directionality of differentiation trajectories (FIG. 2C) (La Manno et al., 2018). In the skeletal muscle trajectory, day 5 mesodermal progenitors gave rise to myogenic progenitor/satellite-like cells (cluster 1,FIG. 2D) which then differentiated through myocytes (cluster 2, FIG. 2D) to contractile skeletal muscle cells (cluster 3, FIG. 2D) (FIGS. 2D and 2E). Indeed, the first signs of muscle maturation appeared after 40-50 days in culture as it was then that the NMOs started contracting in 3D, suggesting the formation of functional networks between spinal cord neurons and skeletal muscles. In the neural trajectory, day 5 neuroectodermal progenitors gave rise to neural progenitors (cluster 4, FIG. 2D), which differentiated through immature neurons (cluster 5,FIG. 2D) into mature spinal cord neurons (cluster 6, FIG. 2D). Additionally, day 5 neuroectodermal cells gave rise to trunk neural crest derivatives (cluster 7, FIG. 2D) as well as glia and Schwann cells (cluster 8, FIG. 2D) (FIGS. 2D and 2F).

To determine whether the different populations were reproducibly generated in different NMOs and batches, all cells of individual batches were clustered and align with the cell populations found in individual NMOs (FIG. 2G). The data show that the cellular composition and developmental trajectories are reproducibly established in each organoid (FIGS. 2G and 2H). Expression of cell type specific neural and muscle markers assayed by immunohistochemistry further confirmed the high consistency among individual organoids at the protein level.

### Organization and Maturation of Trunk NMOs

The separation of the neural and muscle compartments, observed in early NMOs was also maintained at day 50 with clear evidence of extensive neuromuscular interactions. Skeletal muscle fibers were localized in a well-defined region of the organoid that was expressing myosin skeletal fast (Fast MyHC) whereas the neural part was enriched for TUJ1⁺ neurons that were also innervating the muscle region (FIGS. 3A and 3B). Axonal tracts innervating the skeletal muscle were clearly evident by the expression of the mature neurofilament marker SMI32 in the muscle compartment.

To assess reproducibility of the organoids the percentage of neural and muscle populations in at least three individual organoids from three different batches in three different hPSC lines was analyzed (H9, H1 and XM001 hPSCs). The data suggested that NMOs of any given hPSC line reproducibly organize into muscle and neural regions with similar proportions between different batches (FIG. 3C). In agreement with a predisposition of different human PSC lines to favor differentiation into specific germ layers (Osafune et al., 2008) it was observed that while the H9 hPSC line was generating muscle and neural tissue in roughly equal proportions, the H1 and XM001 PSC lines had a slight preference for muscle and neural tissue, respectively (FIG. 3C).

To define the posterior identity of the developing neuromuscular system the expression of HOX genes was examined by single-cell RNA sequencing and immunohistochemistry. The analysis revealed that the NMOs had maintained their initial HOX code corresponding to a posterior spinal cord identity (Dasen et al., 2005; Philippidou and Dasen, 2013). Immunofluorescence analysis for HOXC6 (brachial identity), HOXC9 (thoracic identity) and HOXC10 (posterior thoracic/lumbar identity) revealed that the NMOs have a thoracic/lumbar identity (HOXC6⁻/HOXC9⁺/HOXC10⁺). HOXC10⁺ neurons were expressing the mature neurofilament marker SMI32.

Consistent with continuing maturation of the organoids, most spinal cord neurons were expressing the neurofilament marker SMI32 (75.6%±6.4) while MNs expressing the acetylcholine-synthesizing enzyme choline acetyltransferase (ChAT) (6.4%±1.4) were found clustered in the neural part of the organoid close to the skeletal muscle. Mature spinal cord neurons and MNs were reproducibly generated in similar proportions in NMOs generated by different hPSC lines and experimental batches. Electron microscopy revealed the presence of longitudinally arranged, mitochondria-containing axons, the formation of synaptic clefts and the presence of synaptic vesicles in the presynaptic neuron (FIGS. 3F and 3G), which suggested the formation of functional synapses. GFAP⁺ glia cells (FIG. 4A) as well as myelinated axons detected by the expression of myelin basic protein (MBP) (FIG. 4E and 4F) were also first detected at this stage. Similarly to in vivo development, the number of GFAP⁺ cells increased from day 50 (14.2%±4.1) to day 150 (58.2%±6.8), recapitulating the later developmental timing of glial cells in vivo (FIG. 4A-D).

Day 50 analyses also unveiled the maturation of the muscle part of the organoids. The formation of basal lamina was now evident by the deposition of a continuous sheath of laminin around individual muscle fibers. These muscle fibers were in contact with motor neuron boutons expressing synaptophysin. Electron microscopy revealed the development of highly organized skeletal muscle exhibiting aligned sarcomeric units with the presence of distinct Z and M lines (FIGS. 3D and 3E). Numerous PAX7⁺/Ki67⁺ and PAX7⁺/Ki67⁻ cells were also present in the muscle region suggesting the presence of both proliferating muscle progenitors and satellite-like cells. The location of many PAX7⁺ cells under the basal lamina and the detection of quiescent PAX7⁺/Ki67⁻ cells also supported that satellite-like cells were present in the NMOs.

To further assess this, the number of PAX7⁺ cells and the ratio of mitotically active, PAX7⁺/Ki67⁺ cells was quantified in day 50, day 100 and day 150 organoids. The number of PAX7⁺/Ki67⁻ cells declined steadily from 18.53%±9.17 at day 50 to 8.04%±3.13 at day 150, consistent with continuing maturation of the muscle. The number of PAX7⁺/Ki67⁺ cells also significantly decreased from 0.93%±0.75 at day 50 to 0.06%±0.036 at day 150 suggesting that quiescent PAX7⁺ cells persisted in day 150 month-old organoids. Muscle maturation at day 150 was also documented by the presence of muscle fibers with peripheral nuclei and striations observed with TITIN immunofluorescence analysis (FIG. 4D). Collectively, these data demonstrated muscle development and maturation in the 3D NMOs including the generation of muscle satellite-like cells.

### NMOs Develop Functional NMJs and Model Key Aspects of Myasthenia Gravis.

At day 50 NMOs, numerous, large AChR clusters were detected by staining for α-bungarotoxin (αBTX) contacted by TUJ1⁺ neurites, suggested the formation of NMJs (FIG. 5A). The formation of functional NMJs in 3D was confirmed by the presence of synaptic vesicles in the presynaptic nerve terminal and by the folding of the muscle basement membrane using electron microscopy (FIG. 5D). Strikingly, S100β⁺ terminal Schwann cells capping the neuronal terminals could also be detected (FIG. 5C). Generation of Schwann cells was in agreement with the presence of trunk neural crest cells in early NMOs (Frith et al., 2018). The number of NMJs in organoids from three different human PSC lines and three different batches from each line was analyzed at day 50. The results show that the number of NMJs is similar among different NMOs, batches and hPSC lines (FIG. 5B), which suggested that formation of NMJs in 3D is independent of cell lines and experiments. Finally, and consistent with the physiological pruning process taking place during neuromuscular development (Sanes and Lichtman, 1999), the number of αBTX⁺ clusters, normalized for the number of muscle fibers, initially declined from day 50 to day 100 whereas after that period the number of NMJs was stable (day 150) (FIG. 5B). Thus all the different cell types required for the formation of functional NMJs were generated and self-organized in 3D, resembling the in vivo process.

To investigate the functionality of NMJs, the contractile activity of NMOs was measured before and after treatment with Curare (10 uM), a blocker of AChRs. Treatment with curare (10 uM) blocked the contraction of the skeletal muscles demonstrating that muscle activity was dependent upon functional NMJs.

Next, it was addressed whether the NMOs were suitable to model a common autoimmune disease that affects the NMJ, myasthenia gravis (Toyka et al., 1977). MG is caused by autoantibodies against NMJ specific proteins such as the AChR. The autoimmune attack directed at AChRs results in destruction of the neuromuscular endplate (Sahashi et al, 1980). To this end, the NMOs were treated with purified IgG fractions (300 nM total IgG) from two patients with MG that had high titers of autoantibodies against the AChR (Methods). At least three different organoids were incubated for 72 hours with fresh human serum (2%) containing active complement and specific IgGs from each patient. Quantification of αBTX clusters in the NMOs after treatment revealed a severe reduction in the number of NMJs in the NMOs treated with patient serum compared to controls (FIGS. 5F and 5G). This resulted in a significant reduction of the contraction rate and amplitude in the treated NMOs (FIGS. 5H and 5I). These findings recapitulated key aspects of the disease pathology, suggesting that NMOs reliably model such disorders, which will be useful for future studies.

NMOs form Central Pattern Generator-Like Circuits.

The key physiological parameters in the NMOs were characterized by calcium imaging and electrophysiological analysis using a multi-electrode array system (MEA) (FIG. 6). Calcium oscillations revealed spontaneous neural activity while some neurons were showing synchronous firing at day 50, (FIG. 6D). MEA analysis detected spontaneous electrical activity already from day 30. Acute glutamate treatment (50 uM) significantly increased (approximately 12-fold) the spontaneous activity of day 30 and day 50 organoids, while the specific glutamate receptor inhibitors APV (50 uM) and CNQX (40 uM) reversibly suppressed nearly all electrical activity demonstrating the presence of active glutamatergic neurons. In day 50 NMOs, acetylcholine (10 uM) stimulation also resulted in significant increase (approximately 10-fold) of electrical activity that was rapidly and reversibly blocked by the addition of curare (10 uM). This is consistent with the observed clustering of AChR in the muscle region at this stage (FIG. 5A).

To assess whether functional networks were forming between spinal cord neurons the presence of spinal cord interneurons was first examined. Indeed, at day 50 NMOs the presence of PAX2⁺/LHX1⁺ interneurons (characteristic of V0, V1 and dl6 identity) and CHX10⁺ V2a excitatory pre-motor interneurons could be detected (FIG. 6A-C). To examine the organoid responses to a fictive locomotion paradigm (Marder and Bucher, 2001; Sternfeld et al., 2017), a cocktail of NMDA (10 uM) and 5-HT (40 uM) was applied, which are known to trigger the rhythmic activity of the locomotor central pattern generators located in the spinal cord. Such circuits are essential for a coordinated MN activity (Svensson et al., 2001). Administration of these drugs instantaneously triggered strong activity that was reversibly suppressed by administration of 1 uM tetrodotoxin (TTX), a neurotoxin that inhibits the firing of action potentials. Strikingly, the activity became rhythmic at day 50 NMOs suggesting the maturation of the neural networks and acquisition of central pattern generator-like activity (FIG. 6E-6G).

The activity of these neural networks was analyzed by plotting the number of active electrodes and the corresponding electrical activity before and after administration of the drugs in a network grid. In this grid, node size corresponds to the number of spikes captured by each single electrode during a recording, whereas the thickness of lines connecting the nodes represents the number of times that paired firing between electrodes occurred (FIG. 6G). The position of the individual NMOs in the MEA grid is shown with a dashed line. This analysis demonstrated that stimulation of neurons at day 50 with NMDA and 5-HT resulted in a notable increase of local firings and in a dramatic increase of synchronous network activity (FIG. 6F and 6G).

### Discussion

Neuromuscular diseases encompass a wide range of pathologies such as MN diseases (de Boer et al., 2014; Lefebvre et al., 1995), specific muscular dystrophies (Becker and Kiener, 1955; Pearce et al., 1964) and auto-immune diseases such as Myasthenia gravis (Toyka et al., 1977). In most of these disorders, only the MNs or skeletal muscles are initially affected leading to defects in their interaction, whereas at late stages of the disease also the other cell types are affected. Therefore it becomes apparent that the simultaneous generation of both cell types from hPSC in a 3D organoid model is necessary in order to appropriately model the mechanisms of such diseases, particularly those in which early stages of NMJ formation are impaired.

While organoids for multiple regions of the central nervous system have been established (Birey et al., 2017; Jo et al., 2016; Lancaster et al., 2013; Meinhardt et al., 2014; Ogura et al., 2018; Ranga et al., 2016; Sakaguchi et al., 2015), the generation of a complex, 3D neuromuscular organoid from NMPs has to the inventor's knowledge not been described before. Recently, the in vitro generation of NMPs from hPSCs has been instrumental for the generation of posterior spinal cord neurons and skeletal muscle cells from mouse and human PSCs in conventional monolayer cultures (Chal et al., 2016; Chal et al., 2015; Chal and Pourquie, 2017; Gouti et al., 2014; Lippmann et al., 2015; Maury et al., 2015). Advantage was taken of the developmental potential of NMPs to generate a complex 3D NMO model where two different tissues, spinal cord neurons and skeletal muscles, develop in parallel, self-organize and interact to form functional neuromuscular networks. Such NMOs were generated from different human PSC lines as well as an iPSC line showing that the protocol works reproducibly in different experiments and cell lines.

In NMOs, spinal cord neurons, skeletal muscles and terminal glial cells interact to generate functional NMJs that were analyzed anatomically and functionally. Strikingly, NMOs from all different human PSC lines examined started contracting in 3D between days 40-50 concomitant with the accumulation of αBTX clusters in the muscle fibers. Indeed, blocking of AChR with curare resulted in organoid relaxation suggesting the presence of functional NMJ between MNs and skeletal muscles in 3D. Thus, NMOs contain key-functional components of the neuromuscular system.

Complex interactions between spinal cord neurons occur during development, resulting in the generation of neuronal networks resembling central pattern generators. It has been previously shown that activation of NMDA and Serotonin (5-HT) receptors play an important role in the initiation of locomotor rhythms in mammalian spinal cord but also in artificial neural networks, named “circuitoids” (Sternfeld et al., 2017). Remarkably, it was found that stimulation with NMDA/5-HT produced rhythmic bursts of electrical activity, suggesting the establishment of network connections between interneurons and MNs in the NMOs. Generation of CPG-like networks in human NMOs will allow further studies regarding their involvement in neurodegenerative diseases. Such studies have so far been limited due to the lack of an amenable human model and have been mostly conducted in mouse models and spinal cord explant cultures.

NMOs contain key-functional components of the neuromuscular system, they are highly reproducible and can be easily maintained in culture for several months. These attributes make NMOs an attractive system to study neuromuscular disorders and to develop potential therapies. As a proof of principle, myasthenia gravis, a frequent autoimmune disease that specifically disrupts NMJs and thus affects muscle contraction, was modeled. NMOs treated with autoantibodies from MG patients showed a severe dysfunction of the NMJs and relaxation of the muscle recapitulating key aspects of the disease phenotype. NMOs from iPSC lines of patients with neuromuscular disorders could be used in the future to generate a completely patient derived NMJ model in 3D. This would provide a platform to assess the effectiveness of different pharmacological agents in stabilizing the NMJ and promoting MN survival. It would also give unprecedented access to the early stages of the diseases that precede clinical diagnosis.

2D or 3D co-culture approaches and 3D NMOs such as those developed here can be used in a complementary fashion as they present distinct properties (Pasca, 2018). For example, an important property of the co-culture system is that it allows the generation of specific cell types from different genetic backgrounds, thus addressing mechanism of disease only in one population (for example MNs). On the other hand, 3D cultures can be maintained for longer periods (over one year) and provide access to diverse cell types at different maturation states. Thus NMOs are better suited to study the contribution of each cell type, including terminal Schwann cells, at different stages of NMJ development and maturation that may contribute to the disease phenotype.

An important caveat of 2D co-culture systems has been the lack of functional mature skeletal muscle fibers generated from human PSCs. Most studies relied on the use of primary skeletal muscle from human biopsies or immature muscles generated separately from hPSCs. These approaches lack the timely interaction between MNs and muscle fibers that are necessary for NMJ maturation. In addition, it has been recently shown that maturation of skeletal muscles in 3D is more advanced than the one achieved in 2D culture conditions (Afshar Bakooshli et al., 2019). Crucially, input from the MNs is required for proper muscle maturation and generation of functional NMJs (Machado et al., 2019; Misgeld et al., 2002; Steinbeck et al., 2016). Therefore, in co-culture studies chemical or optogenetic stimulation of MNs has been used to activate the clustering of AChRs and to induce the formation of functional NMJs (Afshar Bakooshli et al., 2019; Machado et al., 2019; Steinbeck et al., 2016). This adds an additional degree of complexity and variability of such systems.

It is important to note here that in the 3D NMO system, clustering of AChR and formation of functional NMJs happens in the absence of exogenous stimuli and in minimal culture conditions because MNs, skeletal muscles and Schwann cells co-develop and interact in the system from its inception. Additionally, NMOs support the contractility of maturing myofibers. Such contractility does not usually develop in 2D co-culture systems, possibly due to disadvantages of the cell culture substrates, which necessitates further studies modifying the 2D microenvironment. Similarly, in the future it will be important to identify the correct time window during which skeletal muscles need to receive input from the MN in order to develop intrinsically functional NMJ networks that do not rely on exogenous stimuli. Conditions that allows the simultaneous generation of MNs, skeletal muscle fibers and terminal Schwann cells in 2D might be useful for this.

Further refinement of the anterior-posterior identity of NMOs to generate all the different segments of the spinal cord and associated musculature could provide novel insights into the selective vulnerability of specific types of MNs to disease. An additional future challenge will be to achieve full maturation of the NMOs by providing input from the motor cortex. Indeed, the ability of cerebral organoids to form extracortical projecting tracts, which innervate and activate mouse spinal cord-muscle explants has been recently described (Giandomenico et al., 2019). Thus, the establishment of NMOs affords the opportunity to establish and study the formation of these complex networks in an all-human in vitro 3D model.

### Experimental Model and Subject Details

### Human Pluripotent Stem Cell Lines and Culture Conditions

The female H9, H9 SOX2^{GFP} and the male H1 human embryonic stem cell lines (obtained from WiCell, and approved for use in this project by the Regulatory Authority for the Import and Use of Human Embryonic Stem Cells in the Robert Koch Institute (AZ:3.04.02/0123) and the female XM001 iPSC line (Wang et al., 2018) were maintained in mTESR1 medium (Stem Cell Technologies) on Geltrex LDEV-Free hESC-Qualified Reduced Growth Factor Basement Membrane Matrix (Life Technologies) at 37° C. Cell lines were checked for normal karyotype and were mycoplasma free. The cells were passaged twice a week using Versene solution (Thermo Fisher).

### Human Samples

Serum from two patients, one male and one female with myasthenia gravis and one healthy male individual, was used with written informed consent from all donors.

### Method Details

In Vitro Generation of NMPs from Human Pluripotent Stem Cells

Human PSCs were grown for at least three passages and after they reached 70% confluency they were dissociated into single cells using accutase. Single cells were counted using the Countess II automated cell counter (Thermo Fisher) and were plated on p35 dishes coated with geltrex (Life Technologies) at a density of 75.000-125.000/cm². The initial plating concentration of the PSCs was adjusted depending on the growth rate of the specific human cell line (XM001: 75.000/cm²; H9 line: 110.000/cm²; H1 line: 100.000/cm²). The first day the cells were plated in neurobasal (NB) medium supplemented with 10 uM ROCK inhibitor (Tocris Bioscience), 3 uM CHIR99021 (Tocris Bioscience) and 40 ng/ml bFGF (Peprotech). NB is a 1:1 medium of Advanced Dulbecco's Modified Eagle Medium (DMEM) comprising Ham's F12 medium supplemented with 1×N2 (Gibco), and Neurobasal medium (Gibco) supplemented with 1×B27 (Gibco), 2 mM L-glutamine (Gibco), 40 ug/ml BSA fraction V (Sigma), 0.1 mM 2-mercaptoethanol (Gibco). The next day ROCK inhibitor was removed and the cells were maintained in NB medium supplemented with 3 uM CHIR99021 and 40 ng/ml bFGF (Peprotech) until day 3. The medium was changed every day. At day 3 the cells were analyzed by immunofluorescence for the co-expression of the NMP markers T/BRA and SOX2.

### Generation of Neuromuscular Organoids in 3D

Neuromesodermal progenitors generated from human PSCs were dissociated using accutase to generate a single cell suspension. On day 0 of organoid formation, NMP cells (4,500-9,000/well depending on the cell line) were plated on an ultra-low binding 96-well plate (Corning) in NB medium with 50 uM Rho-associated protein kinase ROCK inhibitor (Tocris Bioscience), 10 ng/ml bFGF and 2 ng/ml IGF1 and 2 ng/ml HGF (Peprotech). The plates were centrifuged for 2 min at 350 G. The initial volume in each well was 100 ul. At day 2, 50 ul of the medium was removed and 100 ul of NB medium supplemented with 2 ng/ml IGF1 and HGF was added. After day 4 the organoids were maintained in NB medium without the addition of growth factors. On day 10 the organoids were transferred in 60 mm dishes (Corning) in 5 mL of medium and after 1 month in 100 mm dishes (Corning) with 12 mL NB medium. During the whole period organoids were maintained on an orbital shaker rotating at 75 rpm.

### Immunohistochemical Analysis of Organoids

Organoids were fixed with 4% PFA for 30 minutes to 3 hours (depending on size), washed 3 times in PBS and then left overnight in 30% sucrose solution. Organoids were embedded using a 15% gelatin/10% sucrose solution warmed to 42° C. After letting the gelatine/sucrose harden at 4° C., the resulting blocks were frozen in isopentane and stored in −80° C. Organoids were cryosectioned in 10 pm thick slices using a MicroM HM 560 Cryostat (Thermo Fisher) and collected on Superfrost Ultraplus glass slides (Thermo Fisher). The gelatin was removed from the slides by incubating them in PBS at 42° C. for 20 minutes. Slides were blocked in PBS with 4% Bovine Serum Albumin (BSA) (Sigma Aldrich) and 0.3% Triton-X 100 (Sigma Aldrich) for 1 hour at room temperature and incubated with primary antibodies overnight at 4° C. Primary antibodies were washed off 3× with PBS with 0.3% Triton-X 100 (PBST). Slides were incubated with secondary antibodies for 1-2 hours at room temperature. After secondary incubation, slides were washed 3× with PBST and mounted using Fluoroshield Mounting Medium with DAPI (Abcam).

### Reverse Transcription—Quantitative PCR Analysis

Total RNA was isolated from whole organoids or cells growing in monolayer using the RNeasy kit (Qiagen) according to the manufacturer's instructions and digested with DNase I (Qiagen) to remove genomic DNA. First strand cDNA synthesis was performed with Superscript III system (Invitrogen) using random primers and amplified using Platinum SYBR-Green (Invitrogen). For QPCR the Applied Biosystems Quantstudio 6 Flex Real-Time PCR system was used. PCR primers were designed using NCBI Primer-Blast software, using exon-spanning junctions (Table S3). Expression values for each gene were normalized against GAPDH, using the delta-delta CT method and standard deviations were calculated and plotted using Prism 7 software (GraphPad). Error bars represent standard deviation across three biological replicate samples.

### Live Imaging of Organoids

After plating the single cell suspension in an ultra-low attachment round bottom 96-well plate, the cells were transferred to an Incucyte Zoom (Essen Bioscience) live imaging system. Bright-field pictures were acquired of each well in an interval of 15 minutes with 4× zoom for a period of 5 days. Temperature was maintained at 37° C. and CO2 at 5%. Medium changes were performed in between acquisition periods as dictated by the protocol. Images were compressed and exported as an .mp4 movie.

### Electron Microscopy of Organoids

Organoids were fixed with 2% (w/v) paraformaldehyde and 2.5% (v/v) glutaraldehyde in 0.1 M phosphate buffer for 2 hours at room temperature. Samples were postfixed with 1% (v/v) osmium tetroxide, dehydrated in a graded series of ethanol, and embedded in PolyBed® 812 resin (Polysciences, Germany). Ultrathin sections (60-80 nm) were stained with uranyl acetate and lead citrate, and examined at 80 kV with a Zeiss EM 910 electron microscope (Zeiss, Germany). Acquisition was done with a Quemesa CDD camera and the iTEM software (Emsis GmbH, Germany).

Organoids Cell Dissociation for scRNAseq Analysis

Organoids were transferred into a conical tube, washed with PBS and incubated with 1 ml accutase (Life technologies) for 10-20 mins at 37° C. The organoids were mechanically dissociated using a pipette until a single cell suspension was obtained. The cell suspension was run through a 40 μm cell strainer (Miltenyi Biotec) to remove aggregates and debris. A small volume of the cells was used for cell counts and the rest was resuspended in PBS containing 0.04% BSA (Sigma) to give a final concentration of 700 cells/μl.

### Tag Incubation for Single-Cell RNA-Sequencing Analysis

The single cell suspension obtained from organoids was resuspended in 200 μl of BD Stain Buffer (BD Biosciences). Tubes containing the DNA Tags were briefly centrifuged. Each sample (180 μl) was separately transferred to a tube containing a DNA Tag and mixed using a pipette. The mix was left to incubate for 20 minutes at room temperature. Then, 200 μl of BD Stain

Buffer was added to the suspension, and the mix was centrifuged for 5 minutes at 300 g. Supernatant was removed without disturbing the pellet and cells were resuspended in 500 μl Sample Buffer (BD Biosciences). The cells were centrifuged for 5 minutes at 300 g and resuspended in PBS containing 0.04% BSA. The cell suspension was run through a 40 μm cell strainer (Miltenyi Biotec) to remove aggregates and debris. A small volume of the cells was used for cell counts and the rest was resuspended in PBS containing 0.04% BSA (Sigma) to give a final concentration of 700 cells/μl.

### Single-Cell RNA-Sequencing

Single-cell transcriptomics profiling derived from day 5 and day 50 3D organoids was done using the Chromium Single Cell Gene Expression system (10× Genomics), according to the manufacturer's recommendations using the Single Cell 3′ Reagent v2/v3 kits (10× Genomics). Successful library preparation was confirmed using Bioanalyzer device (DNA HS kit, Agilent) and KAPA Library Quantification (KK4857, Roche). Libraries were sequenced using Illumina HiSeq 4000.

Mapping and Extraction of Single-Cell mRNA Transcript Counts

Cell Ranger (v3.0.2) was used to perform barcode processing, mapping and UMI (unique molecular identifier) counting and dimension reduction. Reads were aligned to human GRCh38 reference genome and annotated and counted with gene annotations Ensembl version 93. Final results of the Cell Ranger analysis contain the count values of UMIs assigned to each gene in each of the cells for each respective sample using all mapped reads. The summary of all statistics for each sample and sequencing data discussed in this publication have been deposited in NCBI's Gene Expression Omnibus (Edgar et al., 2002). Data is accessible through GEO Series accession number GSE128357 (www.ncbi.nlm.nih.gov/geo/query/acc.cgi?acc=GSE128357).

### Single-Cell Data Analysis

Further computational analysis was done using Seurat 3.0.3 (Stuart et al., 2019) filtered feature-barcode matrices. Genes expressed in less than 5 cells and cells with less than 500 features expressed were removed. Top 2000 variable genes were calculated using the variance stabilizing transformation (vst) method. Percent of mitochondrial transcripts were calculated and used for visualization. After data integration using pre-computed anchorsets, cell cycle scores were calculated using cell-cycle genes previously described (Tirosh et al., 2016). Cell cycle scores were used to regress out the cell cycle effects before performing principal component analysis (PCA). After running PCA, uniform manifold approximation and projection (Miller et al.) UMAP dimensional reduction was run to visualize cells. Clusters of cells with similar expression patterns were identified by shared nearest neighbor (SNN) modularity optimization based clustering. Visualizations were done using ggplot2 and rgl (Wickham, 2016; Daniel Adler, 2019)

Demultiplexing (cell hashing) of cells from samples was done using oligo-tagged antibodies from BD Single-cell Multiplexing Kit following the BD single cell genomics bioinformatics handbook (bitbucket.org/CRSwDev/multiplexing tools). Numbers of UMI-filtered counts for each sample tag were divided by sample tag-specific means. The sample tag with the highest count per cell was selected to classify its sample origin.

Velocyto.py (La Manno, 2018) annotator was used for each mapped bam file using the default parameters for 10× Genomics technology and the same gtf file for intron-exon annotation. The resulting loom object for each sample was loaded and processed in R using the velocyto.R (v. 0.17) package. The UMAP embeddings from Seurat were used for cell-cell distance calculation and final velocity plots. The estimation of RNA velocity was done with default parameters.

Calcium Imaging of Organoids Calcium imaging was performed on organoid sections. For sectioning, organoids were embedded in low melting point 2% agarose (UltraPure™ Low Melting Point Agarose, Thermofisher) and slices were obtained using a motorized vibratome (Leica, VT1000 S). Two to three consecutive 200 μm section were obtained in iced-cold PBS and immediately transferred to a cell culture insert (Millicell, 0.4 μm, 30 mm diameter, Millipore) placed in 6 well plates. Slices were incubated in NB medium containing 1% fetal bovine serum (Gibco) overnight to allow attachment to the filter before being processed for the calcium imaging experiment.

For calcium imaging experiments 200 μm organoid slices were incubated at 37° C. with 4 uM of the cell permeant calcium indicator Fluo-8 AM (Abcam) supplement with 0.2% DMSO and 0.05% of cremophor to facilitate dye penetration. After 30 minutes of incubation, tissues were washed two times with culture medium (NB without phenol red), placed in the imaging set up and left to recover for 15 min prior to the start of any optical recording. Samples were continuously perfused with heated (37° C.) and oxygenated (Carbogen mix) medium (2 mL/min) during all the experiment.

Fluorescent time series images were acquired with a sCMOS camera (Zyla 4.2 sCMOS, Andor Technology Ltd., Belfast, UK) mounted on an upright microscope (BX51 Olympus, Hamburg, Germany,) using a 10× or 20× water immersion objective and a GFP filter cube set. Images were captured with 100 ms exposure time (10 Hz) and bin size 3×3. Camera control and all post-hoc calcium imaging analysis were performed using the open source Physlmage package implementing Micro-manager software (Hayes et al., 2018). Fluorescent image stacks were processed using “Calmager” filter to obtain a ΔF/F file. A standard-deviation image was generated in order to detect pixels displaying the most variable intensity during the recording. Individual or small cell clusters were automatically identified and the ΔF/F variation over time was plotted for the defined regions of interest. Finally, a cycle trigger average (CTA) was performed to identify cell morphology. In preliminary 2D culture set of experiment it was observed that Fluo-8 AM dye was preferentially taken up by the neuronal population but not myofibers, offering us an adequate method to investigate neural activity.

### Contraction Analysis

Video segments of equal length were recorded using a bright-field microscope (DMI1, Leica) in 3 different regions of the same organoid using 20× zoom. To quantify muscle contractions, each movie was analyzed separately where each visible contraction was counted and then averaged for the whole organoid.

To analyze and map organoid muscular contraction, bright field time series images were automatically thresholded using ImageJ software to identify organoid border. A binary stack was created and contractile regions were visually identified. Regions of interest were defined and variations of organoid area (in pixel), used as a proxy of physical contraction, were plotted over the time series.

### Cell Counting and Statistical Analysis

To quantify the percentage of neurons and muscle in the organoids, TUJ1 expression was used as a neural marker and Myosin Skeletal Fast (MSF) as a marker of skeletal muscle. In z-stacks generated by immunofluorescence analysis, the TUJ1⁺ and MSF⁺ areas were measured separately using ImageJ by defining polygon areas and then normalized to the total area of the organoid sections.

To quantify the percentage of ChAT⁺, GFAP⁺ and SMI32⁺ areas in organoid sections, immunofluorescence z-stacks were used and normalized to the total area of the organoids. To determine and quantify the number of AChR clusters in sections of organoids, α-bungarotoxin staining was performed. Sections of organoids previously fixed were incubated with Alexa Fluor 647 conjugated α-bungarotoxin (Thermo Fisher) for 2 hours to label AChRs. Images were acquired with 80× zoom in 3 random locations per sample (n=3-16). The generated stacks were analyzed using ImageYs Particle Analyzer. Clusters smaller than 5 μm² were excluded from analysis. To assess cluster number, AChR clusters were quantified for each image and then normalized to the number of Myosin Skeletal Fast positive fibers present in the quantified image.

To determine the number of co-localizing cells, stacks acquired with 20× zoom of whole organoid sections were analyzed using Imaris (Bitplane). Each channel was quantified separately. Particles with a diameter of less than 7 μm were excluded from the analysis. Co-localized cells were manually counted and compared to the software analysis. Positive cells were then normalized to the total number of DAPI⁺ cells in the whole section.

### Microelectrode Array (MEA) Recording and Analysis

Whole organoids kept in neurobasal medium were transferred prior to recording to a 6-well MEA plate (M384-tMEA-6W, 64 electrodes, 11.5 μm diameter, 300 μm spacing, Axion Biosystems) and recorded in a Maestro Pro MEA system (Axion Biosystems). Media was removed from each well until only a thin layer remained to promote attachment of organoids to the electrode grid. Temperature was maintained at 37° C. and CO2 at 5%. During some recordings, 50 μM Glutamate or 10 μM ACh or 50 μM NMDA+40 μM 5-HT (Sigma Aldrich) were added in warm media to stimulate organoid activity. To record inhibitory pharmacologic effects on organoids, 10 μM Curare or 50 μM APV and 40 μM CNQX (Sigma Aldrich) or 1 μM Tetrodotoxin (Abcam) were added to the medium. After inhibitory recordings the organoids were washed with warm basal medium and left to stabilize for 30 minutes, after which a new recording was performed. The signal was sampled at 25 kHz and stored using the AXIS Navigator Software (Axion Biosystems). Data was exported as .csv files for analysis. A threshold 6 times the standard deviation above the background noise was used to detect extracellular spikes in each channel with a 2 ms refractory period imposed after each detected spike. Connectivity between electrodes was determined within a time window of 40 ms. Analysis of the resulting connectivity data was performed using a Matlab script where all spikes were counted for each single electrode and a combinatorial non-repetitive description of each paired electrode connections was obtained and plotted.

### Myasthenia Gravis Disease Modelling

Serum from two patients diagnosed with Anti-AChR MG having high antibody titters (Patient 1>20; Patient 2=7.6) was collected and IgG fractions were purified using a Protein G Serum Antibody Purification Kit (Abcam) according to the manufacturer's instructions.

Purified IgG was reconstituted using the Kit Elution Buffer and IgG content was quantified using a Nanodrop 1000 spectrophotometer (Thermo Fisher). Contracting NMOs were incubated with MG patient IgGs (300 nM final concentration) supplemented with 2% human serum for 3 days. IgGs from healthy human serum (Sigma) was used as a control. The medium was changed every day, and after 3 days of treatment, the NMOs were collected for fixation and immunofluorescence analysis. Analysis of the contractile activity of at least 3 different NMOs was performed before and after treatment (3 days) with specific IgGs. Video segments of 5 minutes in length were recorded using a Leica SP8 confocal, and exported as .avi files. 3 different close-up areas of each NMO were recorded and averaged. Number of muscle contractions were obtained by isolating one visually recognizable contractile area of the video recording using ImageJ (as described previously). The number of peaks resulting from area displacement during contraction were counted and averaged.

### Quantification and Statistical Analysis

Data are reported as the mean±standard deviation, using a significance level of p<0.05. The number of replicates is indicated in the figure legends; “N” denotes the number of independent experiments and “n” denotes the number of organoids, as appropriate. Data were analyzed by one-way and two-way ANOVA, using Bonferroni test for multiple comparisons and Welch's t test for pairwise comparisons (Prism 5-7, GraphPad).

### Data and Software Availability

The single cell data have been deposited in the gene expression omnibus (GEO) under ID code GSE128357 (www.ncbi.nlm.nih.gov/geo/query/acc.cgi?acc=GSE128357).

### Disease Model

FIG. 7A to 7C relate to a disease model for studying spinal muscular atrophy. NMOs were generated from induced pluripotent stem cells of a patient suffering from spinal muscular atrophy. The general scheme of this experiment can be seen from FIG. 7A. 50 days after cultivation start, NMOs were successfully obtained (FIG. 7B). This can be seen by a typical neuromuscular organization of skeletal muscle (stained in green by anti-myosin skeletal fast antibody) and neuronal cells (stained in red by the pan neural marker tubb3 (Tuj1).

Immunostaining with markers for neuromuscular junctions shows smaller muscle fibers and reduced clustering of acetylcholine receptors in disease NMOs (first and second panel from the left of FIG. 7C) compared to wild type NMOs (third and fourth panel from the left of FIG. 7C). A quantitative analysis of the number of neuromuscular junctions (NMJs) per muscle fiber in day 50 NMOs generated from either a human pluripotent stem cell (PSC) line (H9, H1), a wild type induced PSC line (XM001) or an SMN1 mutant induced PSC line (SMAE1C4) is depicted in the very right panel of FIG. 7C.

FIGS. 8A and 8B relate to a disease model for studying amyotrophic lateral sclerosis (ALS). Induced pluripotent stem cells from a patient suffering from amyotrophic lateral sclerosis and an isogenic line with the mutation corrected (Kiskinis et al., 2014) were used to generate neuromuscular organoids. 50 days after cultivation start, NMOs were successfully obtained (FIG. 8B). This can be seen by a typical neuromuscular organization of skeletal muscle (stained in red by anti-myosin skeletal fast antibody) and neuronal cells (stained in green by the pan neural marker tubb3 (Tuj1) and alpha Bungarotoxin (aBTX) in cerulean. The diseased organoids develop spinal cord neurons and skeletal muscles that self-organize to generate NMJs.

FIG. 9A to 9C relate to the long-term maintenance of neuromuscular organoids in culture. The neuromuscular organoids were maintained in culture for 300 days and analyzed by immunofluorescence analysis. The organoids were intact without any signs of deterioration and they had a typical neuromuscular organization of skeletal muscle (stained in green by anti-myosin skeletal fast antibody), neuronal cells (stained in red by the pan neural marker tubb3 (Tuj1) and alpha Bungarotoxin (aBTX) in cerulean. Motor neurons that expressed the neurofilament marker (SMI-32) and Choline Acetyltransferase (CHAT) were present after 300 days in culture. Spinal cord neurons, skeletal muscles and NMJs are present after long term culture, while the organoids still contract.

### REFERENCES CITED IN THE PRECEDING SECTIONS

1. Afshar Bakooshli, M., Lippmann, E. S., Mulcahy, B., Iyer, N., Nguyen, C. T., Tung, K., Stewart, B. A., van den Dorpel, H., Fuehrmann, T., Shoichet, M., et al. (2019). A 3D culture model of innervated human skeletal muscle enables studies of the adult neuromuscular junction. Elife 8.

2. Becht, E., McInnes, L., Healy, J., Dutertre, C. A., Kwok, I. W. H., Ng, L. G., Ginhoux, F., and Newell, E.W. (2018). Dimensionality reduction for visualizing single-cell data using UMAP. Nat Biotechnol.

3. Becker, P. E., and Kiener, F. (1955). [A new x-chromosomal muscular dystrophy]. Arch Psychiatr Nervenkr Z Gesamte Neurol Psychiatr 193, 427-448.

4. Birey, F., Andersen, J., Makinson, C. D., Islam, S., Wei, W., Huber, N., Fan, H. C., Metzler, K. R. C., Panagiotakos, G., Thom, N., et al. (2017). Assembly of functionally integrated human forebrain spheroids. Nature 545, 54-59.

5. Brassard, J. A., and Lutolf, M. P. (2019). Engineering Stem Cell Self-organization to Build Better Organoids. Cell Stem Cell 24, 860-876.

6. Broutier, L., Andersson-Rolf, A., Hindley, C. J., Boj, S. F., Clevers, H., Koo, B. K., and Huch, M. (2016). Culture and establishment of self-renewing human and mouse adult liver and pancreas 3D organoids and their genetic manipulation. Nat Protoc 11, 1724-1743.

7. Cambray, N., and Wilson, V. (2002). Axial progenitors with extensive potency are localised to the mouse chordoneural hinge. Development 129, 4855-4866.

8. Chal, J., Al Tanoury, Z., Hestin, M., Gobert, B., Aivio, S., Hick, A., Cherrier, T., Nesmith, A. P., Parker, K. K., and Pourquie, O. (2016). Generation of human muscle fibers and satellite-like cells from human pluripotent stem cells in vitro. Nat Protoc 11, 1833-1850.

9. Chal, J., Oginuma, M., Al Tanoury, Z., Gobert, B., Sumara, O., Hick, A., Bousson, F., Zidouni, Y., Mursch, C., Moncuquet, P., et al. (2015). Differentiation of pluripotent stem cells to muscle fiber to model Duchenne muscular dystrophy. Nat Biotechnol 33, 962-969.

10. Chal, J., and Pourquie, O. (2017). Making muscle: skeletal myogenesis in vivo and in vitro. Development 144, 2104-2122.

11. Darabid, H., Perez-Gonzalez, A. P., and Robitaille, R. (2014). Neuromuscular synaptogenesis: coordinating partners with multiple functions. Nat Rev Neurosci 15, 703-718.

12. Dasen, J. S., Tice, B. C., Brenner-Morton, S., and Jessell, T. M. (2005). A Hox regulatory network establishes motor neuron pool identity and target-muscle connectivity. Cell 123, 477-491.

13. de Boer, A. S., Koszka, K., Kiskinis, E., Suzuki, N., Davis-Dusenbery, B. N., and Eggan, K. (2014). Genetic validation of a therapeutic target in a mouse model of ALS. Sci Transl Med 6, 248ra104.

14. Deschamps, J., and Duboule, D. (2017). Embryonic timing, axial stem cells, chromatin dynamics, and the Hox clock. Genes Dev 31, 1406-1416.

15. Edgar, R., Domrachev, M., and Lash, A. E. (2002). Gene Expression Omnibus: NCBI gene expression and hybridization array data repository. Nucleic Acids Res 30, 207-210.

16. Eiraku, M., and Sasai, Y. (2012). Self-formation of layered neural structures in three-dimensional culture of ES cells. Curr Opin Neurobiol 22, 768-777.

17. Forlani, S., Lawson, K. A., and Deschamps, J. (2003). Acquisition of Hox codes during gastrulation and axial elongation in the mouse embryo. Development 130, 3807-3819.

18. Frith, T. J., Granata, I., Wind, M., Stout, E., Thompson, O., Neumann, K., Stavish, D., Heath, P. R., Ortmann, D., Hackland, J. O., et al. (2018). Human axial progenitors generate trunk neural crest cells in vitro. Elife 7.

19. Giandomenico, S. L., Mierau, S. B., Gibbons, G. M., Wenger, L. M. D., Masullo, L., Sit, T., Sutcliffe, M., Boulanger, J., Tripodi, M., Derivery, E., et al. (2019). Cerebral organoids at the air-liquid interface generate diverse nerve tracts with functional output. Nat Neurosci 22, 669-679.

20. Gouti, M., Delile, J., Stamataki, D., Wymeersch, F. J., Huang, Y., Kleinjung, J., Wilson, V., and Briscoe, J. (2017). A Gene Regulatory Network Balances Neural and Mesoderm Specification during Vertebrate Trunk Development. Dev Cell 41, 243-261 e247.

21. Gouti, M., Tsakiridis, A., Wymeersch, F. J., Huang, Y., Kleinjung, J., Wilson, V., and Briscoe, J. (2014). In vitro generation of neuromesodermal progenitors reveals distinct roles for wnt signalling in the specification of spinal cord and paraxial mesoderm identity. PLoS Biol 12, e1001937.

22. Henrique, D., Abranches, E., Verrier, L., and Storey, K. G. (2015). Neuromesodermal progenitors and the making of the spinal cord. Development 142, 2864-2875.

23. Huch, M., Gehart, H., van Boxtel, R., Hamer, K., Blokzijl, F., Verstegen, M. M., Ellis, E., van Wenum, M., Fuchs, S. A., de Ligt, J., et al. (2015). Long-term culture of genome-stable bipotent stem cells from adult human liver. Cell 160, 299-312.

24. Jo, J., Xiao, Y., Sun, A. X., Cukuroglu, E., Tran, H. D., Goke, J., Tan, Z. Y., Saw, T. Y., Tan, C. P., Lokman, H., et al. (2016). Midbrain-like Organoids from Human Pluripotent Stem Cells Contain Functional Dopaminergic and Neuromelanin-Producing Neurons. Cell Stem Cell 19, 248-257.

25. Kimelman, D. (2016). Tales of Tails (and Trunks): Forming the Posterior Body in Vertebrate Embryos. Curr Top Dev Biol 116, 517-536.

26. La Manno, G., Soldatov, R., Zeisel, A., Braun, E., Hochgerner, H., Petukhov, V., Lidschreiber, K., Kastriti, M. E., Lonnerberg, P., Furlan, A., et al. (2018). RNA velocity of single cells. Nature 560, 494-498.

27. Lancaster, M. A., Renner, M., Martin, C. A., Wenzel, D., Bicknell, L. S., Hurles, M. E., Homfray, T., Penninger, J. M., Jackson, A. P., and Knoblich, J. A. (2013). Cerebral organoids model human brain development and microcephaly. Nature 501, 373-379.

28. Lefebvre, S., Burglen, L., Reboullet, S., Clermont, O., Burlet, P., Viollet, L., Benichou, B., Cruaud, C., Millasseau, P., Zeviani, M., et al. (1995). Identification and characterization of a spinal muscular atrophy-determining gene. Cell 80, 155-165.

29. Lippmann, E. S., Williams, C. E., Ruhl, D. A., Estevez-Silva, M. C., Chapman, E. R., Coon, J. J., and Ashton, R. S. (2015). Deterministic HOX patterning in human pluripotent stem cell-derived neuroectoderm. Stem Cell Reports 4, 632-644.

30. Machado, C. B., Pluchon, P., Harley, P., Rigby, M., Gonzalez Sabater, V., Stevenson, D. C., Hynes, S., Lowe, A., Burrone, J., Viasnoff, V., et al. (2019). In Vitro Modelling of Nerve-Muscle Connectivity in a Compartmentalised Tissue Culture Device. Adv Biosyst 3.

31. Maffioletti, S. M., Sarcar, S., Henderson, A. B. H., Mannhardt, I., Pinton, L., Moyle, L. A., Steele-Stallard, H., Cappellari, O., Wells, K. E., Ferrari, G., et al. (2018). Three-Dimensional Human iPSC-Derived Artificial Skeletal Muscles Model Muscular Dystrophies and Enable Multilineage Tissue Engineering. Cell Rep 23, 899-908.

32. Marder, E., and Bucher, D. (2001). Central pattern generators and the control of rhythmic movements. Curr Biol 11, R986-996.

33. Maury, Y., Come, J., Piskorowski, R. A., Salah-Mohellibi, N., Chevaleyre, V., Peschanski, M., Martinat, C., and Nedelec, S. (2015). Combinatorial analysis of developmental cues efficiently converts human pluripotent stem cells into multiple neuronal subtypes. Nat Biotechnol 33, 89-96.

34. Meinhardt, A., Eberle, D., Tazaki, A., Ranga, A., Niesche, M., Wilsch-Brauninger, M., Stec, A., Schackert, G., Lutolf, M., and Tanaka, E. M. (2014). 3D reconstitution of the patterned neural tube from embryonic stem cells. Stem Cell Reports 3, 987-999.

35. Metzis, V., Steinhauser, S., Pakanavicius, E., Gouti, M., Stamataki, D., Ivanovitch, K., Watson, T., Rayon, T., Mousavy Gharavy, S. N., Lovell-Badge, R., et al. (2018). Nervous System Regionalization Entails Axial Allocation before Neural Differentiation. Cell 175, 1105-1118 e1117.

36. Miller, T. M., Kim, S. H., Yamanaka, K., Hester, M., Umapathi, P., Arnson, H., Rizo, L., Mendell, J. R., Gage, F. H., Cleveland, D. W., et al. (2006). Gene transfer demonstrates that muscle is not a primary target for non-cell-autonomous toxicity in familial amyotrophic lateral sclerosis. Proc Natl Acad Sci U S A 103, 19546-19551.

37. Misgeld, T., Burgess, R. W., Lewis, R. M., Cunningham, J. M., Lichtman, J. W., and Sanes, J. R. (2002). Roles of neurotransmitter in synapse formation: development of neuromuscular junctions lacking choline acetyltransferase. Neuron 36, 635-648.

38. Morizane, R., Lam, A. Q., Freedman, B. S., Kishi, S., Valerius, M. T., and Bonventre, J. V. (2015). Nephron organoids derived from human pluripotent stem cells model kidney development and injury. Nat Biotechnol 33, 1193-1200.

39. Ogura, T., Sakaguchi, H., Miyamoto, S., and Takahashi, J. (2018). Three-dimensional induction of dorsal, intermediate and ventral spinal cord tissues from human pluripotent stem cells. Development 145.

40. Olivera-Martinez, I., Harada, H., Halley, P. A., and Storey, K. G. (2012). Loss of FGF-dependent mesoderm identity and rise of endogenous retinoid signalling determine cessation of body axis elongation. PLoS Biol 10, e1001415.

41. Osafune, K., Caron, L., Borowiak, M., Martinez, R. J., Fitz-Gerald, C. S., Sato, Y., Cowan, C. A., Chien, K. R., and Melton, D. A. (2008). Marked differences in differentiation propensity among human embryonic stem cell lines. Nat Biotechnol 26, 313-315.

42. Osaki, T., Uzel, S. G. M., and Kamm, R. D. (2018). Microphysiological 3D model of amyotrophic lateral sclerosis (ALS) from human iPS-derived muscle cells and optogenetic motor neurons. Sci Adv 4, eaat5847.

43. Pasca, A. M., Sloan, S. A., Clarke, L. E., Tian, Y., Makinson, C. D., Huber, N., Kim, C. H., Park, J. Y., O'Rourke, N. A., Nguyen, K. D., et al. (2015). Functional cortical neurons and astrocytes from human pluripotent stem cells in 3D culture. Nat Methods 12, 671-678.

44. Pasca, S. P. (2018). The rise of three-dimensional human brain cultures. Nature 553, 437-445.

45. Pearce, J. M., Pennington, R. J., and Walton, J. N. (1964). Serum Enzyme Studies in Muscle Disease. Iii. Serum Creatine Kinase Activity in Relatives of Patients with the Duchenne Type of Muscular Dystrophy. J Neurol Neurosurg Psychiatry 27, 181-185.

46. Philippidou, P., and Dasen, J. S. (2013). Hox genes: choreographers in neural development, architects of circuit organization. Neuron 80, 12-34.

47. Ranga, A., Girgin, M., Meinhardt, A., Eberle, D., Caiazzo, M., Tanaka, E. M., and Lutolf, M. P. (2016). Neural tube morphogenesis in synthetic 3D microenvironments. Proc Natl Acad Sci USA 113, E6831-E6839.

48. Rowe, R. G., and Daley, G. Q. (2019). Induced pluripotent stem cells in disease modelling and drug discovery. Nat Rev Genet.

49. Sakaguchi, H., Kadoshima, T., Soen, M., Narii, N., Ishida, Y., Ohgushi, M., Takahashi, J., Eiraku, M., and Sasai, Y. (2015). Generation of functional hippocampal neurons from self-organizing human embryonic stem cell-derived dorsomedial telencephalic tissue. Nat Commun 6, 8896.

50. Sanes, J. R., and Lichtman, J. W. (1999). Development of the vertebrate neuromuscular junction. Annu Rev Neurosci 22, 389-442.

51. Santhanam, N., Kumanchik, L., Guo, X., Sommerhage, F., Cai, Y., Jackson, M., Martin, C., Saad, G., McAleer, C. W., Wang, Y., et al. (2018). Stem cell derived phenotypic human neuromuscular junction model for dose response evaluation of therapeutics. Biomaterials 166, 64-78.

52. Spence, J. R., Mayhew, C. N., Rankin, S. A., Kuhar, M. F., Vallance, J. E., Tolle, K., Hoskins, E. E., Kalinichenko, V. V., Wells, S. I., Zorn, A. M., et al. (2011). Directed differentiation of human pluripotent stem cells into intestinal tissue in vitro. Nature 470, 105-109.

53. Steinbeck, J. A., Jaiswal, M. K., Calder, E. L., Kishinevsky, S., Weishaupt, A., Toyka, K. V., Goldstein, P. A., and Studer, L. (2016). Functional Connectivity under Optogenetic Control Allows Modeling of Human Neuromuscular Disease. Cell Stem Cell 18, 134-143.

54. Sternfeld, M. J., Hinckley, C. A., Moore, N. J., Pankratz, M. T., Hilde, K. L., Driscoll, S. P., Hayashi, M., Amin, N. D., Bonanomi, D., Gifford, W. D., et al. (2017). Speed and segmentation control mechanisms characterized in rhythmically-active circuits created from spinal neurons produced from genetically-tagged embryonic stem cells. Elife 6.

55. Stoeckius, M., Zheng, S., Houck-Loomis, B., Hao, S., Yeung, B. Z., Mauck, W. M., 3rd, Smibert, P., and Satija, R. (2018). Cell Hashing with barcoded antibodies enables multiplexing and doublet detection for single cell genomics. Genome Biol 19, 224.

56. Stuart, T., Butler, A., Hoffman, P., Hafemeister, C., Papalexi, E., Mauck, W. M., 3rd, Hao, Y., Stoeckius, M., Smibert, P., and Satija, R. (2019). Comprehensive Integration of Single-Cell Data. Cell 177, 1888-1902 e1821.

57. Svensson, E., Grillner, S., and Parker, D. (2001). Gating and braking of short- and long-term modulatory effects by interactions between colocalized neuromodulators. J Neurosci 21, 5984-5992.

58. Tirosh, I., Venteicher, A. S., Hebert, C., Escalante, L. E., Patel, A. P., Yizhak, K., Fisher, J. M., Rodman, C., Mount, C., Filbin, M. G., et al. (2016). Single-cell RNA-seq supports a developmental hierarchy in human oligodendroglioma. Nature 539, 309-313.

59. Toyka, K. V., Drachman, D. B., Griffin, D. E., Pestronk, A., Winkelstein, J. A., Fishbeck, K. H., and Kao, I. (1977). Myasthenia gravis. Study of humoral immune mechanisms by passive transfer to mice. N Engl J Med 296, 125-131.

60. Tzouanacou, E., Wegener, A., Wymeersch, F. J., Wilson, V., and Nicolas, J. F. (2009). Redefining the progression of lineage segregations during mammalian embryogenesis by clonal analysis. Dev Cell 17, 365-376.

61. Verrier, L., Davidson, L., Gierlinski, M., Dady, A., and Storey, K. G. (2018). Neural differentiation, selection and transcriptomic profiling of human neuromesodermal progenitor-like cells in vitro. Development 145.

62. Wang, X., Sterr, M., Burtscher, I., Chen, S., Hieronimus, A., Machicao, F., Staiger, H., Haring, H. U., Lederer, G., Meitinger, T., et al. (2018). Genome-wide analysis of PDX1 target genes in human pancreatic progenitors. Mol Metab 9, 57-68.

63. Wilson, V., Olivera-Martinez, I., and Storey, K. G. (2009). Stem cells, signals and vertebrate body axis extension. Development 136, 1591-1604.

64. Price, Paul J., and Gregory J. Brewer. “Serum-free media for neural cell cultures.” Protocols for neural cell culture. Humana Press, 2001. 255-264.

65. Kiskinis, E., Sandoe, J., Williams, L. A., Boulting, G. L., Moccia, R., Wainger, B. J., Han, S., Peng, T., Thams, S., Mikkilineni, S., et al. (2014). Pathways disrupted in human ALS motor neurons identified through genetic correction of mutant SOD1. Cell Stem Cell 14, 781-795.

66. Martins, J. M. F., Fischer, C., Urzi, A., Vidal, R., Kunz, S., Ruffault, P. L., . . . & Gouti, M. (2020). Self-organizing 3D human trunk neuromuscular organoids. Cell stem cell, 26(2), 172-186.

1. A method for generating a three-dimensional neuromuscular organoid in vitro, comprising:
a) providing a first cell culture comprising disease-specific neuromesodermal progenitor cells, wherein 30% to 90% of the disease-specific neuromesodermal progenitor cells co-express BRACHYURY/SOX2 and 10% to 70% of the disease-specific neuromesodermal progenitor cells co-express TBX6, and cultivating the disease-specific neuromesodermal progenitor cells in a first differentiation medium chosen from the group consisting of i) a non-supplemented serum-free cell culture medium and ii) a serum-free cell culture medium supplemented with at least one of a ROCK inhibitor, an activator of a growth factor signaling pathway, and an activator of an insulin signaling pathway;
b) replacing the first differentiation medium by a second differentiation medium within 1 to 4 days after cultivation start, wherein the second differentiation medium is chosen from the group consisting of i) a non-supplemented serum-free cell culture medium and ii) a serum-free cell culture medium supplemented with at least one of an activator of a growth factor signaling pathway, and an activator of an insulin signaling pathway;
c) replacing the second differentiation medium by a non-supplemented serum-free cell culture medium within 1 to 4 days after replacing the first differentiation medium by the second differentiation medium; and
d) obtaining a three-dimensional neuromuscular organoid from the non-supplemented serum-free cell culture medium.

2. The method according to claim 1, wherein the non-supplemented serum-free cell culture medium is at least one of Dulbecco's Modified Eagle Medium comprising Ham's F12 medium, Advanced Dulbecco's Modified Eagle Medium comprising Ham's F12 medium, neurobasal medium or neurobasal plus medium.

3. The method according to claim 1, wherein the activator of a growth factor signaling pathway is chosen from the group consisting of:
an activator of the fibroblast growth factor signaling pathway,
an activator of the hepatocyte growth factor signaling pathway,
an activator of the insulin-like growth factor signaling pathway, and
an activator of the epidermal growth factor signaling pathway.

4. The method according to claim 1, wherein the first differentiation medium is a serum-free cell culture medium supplemented with a ROCK inhibitor, basic fibroblast growth factor, insulin-like growth factor, and hepatocyte growth factor.

5. The method according to claim 1, wherein the second differentiation medium is a serum-free cell culture medium supplemented with insulin-like growth factor and hepatocyte growth factor.

6. The method according to claim 1, wherein the non-supplemented serum-free cell culture medium is changed after step c) every 1 to 3 days during a period of 10 days after cultivation start and every 2 to 5 days during a period exceeding 10 days after cultivation start.

7. The method according to claim 1, wherein the method is carried out under agitation.

8. The method according to claim 1, wherein the disease-specific neuromesodermal progenitor cells are obtained by providing a second cell culture comprising pluripotent stem cells and cultivating the pluripotent stem cells in a first cultivation medium comprising a serum-free cell culture medium supplemented with at least one of a ROCK inhibitor, an activator of β-catenin signaling pathway, and an activator of a growth factor signaling pathway.

9. The method according to claim 8, wherein first cultivation medium comprises a serum-free cell culture medium supplemented at least with an activator of β-catenin signaling pathway.

10. The method according to claim 8, wherein the activator of β-catenin signaling pathway is an inhibitor of glycogen synthase kinase 3 activity or an activator of Wnt signaling pathway that upregulates an expression of β-catenin.

11. The method according to claim 8, wherein the activator of β-catenin signaling pathway is at least one of the group consisting of:
6-[[2-[[4-(2,4-Dichlorophenyl)-5-(5-methyl-1H-imidazol-2-yl)-2-pyrimidinyl]amino]ethyl]amino]-3-pyridinecarbonitrile,
N-6-[2-[[4-(2,4-Dichlorophenyl)-5-(1H-imidazol-1-yl)-2-pyrimidinyl]amino]ethyl]-3-nitro-2,6-pyridine-diamine,
BIO-acetoxime,
dynein light intermediate chain 1,
3-[(3-Chloro-4-hydroxyphenyl)-amino]-4-(2-nitrophenyl)-1H-pyrrol-2,5-di one,
N-(4-Methoxybenzyl)-N′-(5-nitro-1,3-thiazol-2-yl)urea,
1-azakenpaullone, and
bis-7-indolyl maleimide.

12. The method according to claim 8, wherein the activator of a growth factor signaling pathway is chosen from the group consisting of:
an activator of the fibroblast growth factor signaling pathway,
an activator of the hepatocyte growth factor signaling pathway,
an activator of the insulin-like growth factor signaling pathway,
an activator of the epidermal growth factor signaling pathway,
an activator of the nerve growth factor pathway, and
an activator of the platelet-derived growth factor signaling pathway.

13. The method according to claim 8, wherein the activator of a growth factor signaling pathway is a fibroblast growth factor.

14. The method according to claim 8, wherein:
the first cultivation medium is changed against a second cultivation medium 1 to 3 days after cultivation start, and
the second cultivation medium includes a serum-free cell culture medium supplemented with at least one of an activator of β-catenin signaling pathway and an activator of a growth factor signaling pathway.

15. The method according to claim 14, wherein the second cultivation medium corresponds to the first cultivation medium,. except that the first cultivation medium contains a ROCK inhibitor, but the second cultivation medium does not contain a ROCK inhibitor.

16. A neuromuscular organoid obtainable by a method according to claim 1.

17. The neuromuscular organoid according to claim 16, wherein the neuromuscular organoid does not comprise a vascular system.

18. A method of studying the development and/or mechanism of a disease in vitro, comprising subjecting a neuromuscular organoid according to claim 16 to a reagent and observing an effect of the reagent.

19. A method of studying the development and/or mechanism of a disease in vitro, comprising:
comparing a first neuromuscular organoid according to claim 16 made from a first type of neuromesodermal progenitor cells having a first genetic constitution with a second neuromuscular organoid according to claim 16 made from a second type of neuromesodermal progenitor cells having a second genetic constitution, wherein the second genetic constitution differs from the first genetic constitution.

20. The method according to claim 18, wherein the disease is:
a motor-neuron disease,
a neuromuscular disease,
a rare neuromuscular disease,
a disease affecting the central nervous system of a patient,
a disease affecting the muscular or neuromuscular system of a patient,
a myopathy, or
an auto-immune neuromuscular disease.

21. The method according to claim 20, wherein:
the motor-neuron disease is amyotrophic lateral sclerosis, spinal bulbar muscular atrophy, or spinal muscular atrophy and/or wherein the myopathy is Becker's muscular dystrophy, Duchenne's muscular dystrophy, congenital muscular dystrophy, distal muscular dystrophy, Limb-girdle muscular dystrophy, Emery-Dreifuss muscular dystrophy, Charcot-Marie-Tooth disease, a mitochondrial myopathy, a congenital or a distal myopathy, and/or
the auto-immune neuromuscular disease is myasthenia gravis, and/or
the disease affecting the muscular or neuromuscular system of a patient is giant axonal neuropathy, a congenital myasthenic syndrome, or Lambert-Eaton myasthenic syndrome.

22. (canceled)

23. (canceled)

24. The method according to claim 19, wherein the disease is:
a motor-neuron disease,
a neuromuscular disease,
a rare neuromuscular disease,
a disease affecting the central nervous system of a patient,
a disease affecting the muscular or neuromuscular system of a patient,
a myopathy, or
an auto-immune neuromuscular disease.

25. The method according to claim 24, wherein:
the motor-neuron disease is amyotrophic lateral sclerosis, spinal bulbar muscular atrophy, or spinal muscular atrophy and/or wherein the myopathy is Becker's muscular dystrophy, Duchenne's muscular dystrophy, congenital muscular dystrophy, distal muscular dystrophy, Limb-girdle muscular dystrophy, Emery-Dreifuss muscular dystrophy, Charcot-Marie-Tooth disease, a mitochondrial myopathy, a congenital or a distal myopathy, and/or
the auto-immune neuromuscular disease is myasthenia gravis, and/or the disease affecting the muscular or neuromuscular system of a patient is giant axonal neuropathy, a congenital myasthenic syndrome, or Lambert-Eaton myasthenic syndrome.
